(19) United States
(12) Patent Application Publication
(10) Pub. No.: US 2003/0216318 A1
(43) Pub. Date: Nov. 20, 2003

(54) Inhibition of transmission of tick-borne infections

(76) Inventors: Deborah C. Jaworski, Norco, CA (US); Alan G. Barbour, Newport Beach, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/257,730

(22) PCT Filed: Apr. 13, 2001
(86) PCT No.: PCT/US01/12189

(51) Int. Cl. A61K 38/10; C07K 14/16; C07H 21/04

(52) U.S. Cl. 514/13; 530/326; 536/23.1

(57) ABSTRACT

The present invention is based on the discovery of an arthropod polypeptide which is a homologue of Macrophage Migration Inhibitory Factor (MIF). The present invention relates to the identification and characterization of a homologue of the proinflammatory cytokine, Macrophage Migration Inhibitory Factor in the tick, Amblyomma americanum. The invention provides MIF polypeptide, polynucleodites, antibodies that bind to MIF and methods of use for inducing immunity to ticks, thereby reducing the incidence of tick-borne infections in animals. It should be understood that immunity may also be induced to other species of ticks, including Haemaphysalis spp, Otobius spp, Rhiphicephalus spp, other Ambylomma spp, Dermacentor spp, Ixodes spp and Hyalomma spp and species of Boophilus.

### FIELD OF THE INVENTION

The invention relates generally to cytokines and immune responses and, more specifically, to a proinflammatory cytokine, Macrophage Migration inhibitory Factor (MIF), identified in the tick, Amblyomma americanum.

### BACKGROUND OF THE INVENTION

Ticks are blood-feeding ectoparasites and the vectors of a variety of viral, bacterial, and eukaryotic pathogens of humans and other vertebrates. Among the hard-bodied or ixodid group of ticks, species of the genus Amblyomma transmit several infections of importance to livestock, wildlife, and humans, especially on the African continent. In the United States, the lone star tick, Amblyomma americanum, is the vector of human monocytic erlichiosis and a Lyme disease-like disorder (Walker et al. 1996). Unlike mosquitoes and other arthropod vectors of human diseases, ticks feedonly on blood at each life stage of larva, nymph, and adult. Generally, a single blood meal at each stage provides sustenance, for development to the next life stage, and for egg production. For some pathogens, such as the agent of Lyme disease, the migration of the pathogen from the midgut to the salivary glands requires a blood meal by the tick (de Silva & Fikrig 1995). Another difference between ixodid ticks and mosquitoes is a feeding period easured in days not minutes. During a feeding, ticks consume milliliters of blood hile embedded in the skin and are susceptible to the host's innate and adaptive immune responses. Acquired immunity to tick feeding occurs; repeated tick feedings on the same mammalian host over time lead to reductions in size of the blood meal and the numbers of eggs laid and larvae hatched (Trager 1939; Wikel 1996). The defenses of ticks against the host's immune responses have not been well characterized (Barriga 1999). A number of components of tick saliva have been identified (Nuttall 1998; Ribeiro 1987; Sauer et al. 1994; Sonenshine 1991a), and some of these may modulate or counteract the host's innate and adaptive immune responses (Wikel & Bergman 1997). Cytokine-like components have not be found. However, defining the specific factors involved in evasion of host responses and facilitation of tick feeding has been hampered by the complexity of tick-host interactions. For instance, inhibition of inflammation at the feeding site may limit access of host effector cells to the feeding tick. On the other hand, enhancement of inflammation may increase the amount of blood and fluid at the feeding site.

A large number of approaches are used to control ticks. The most widely used is treatment of cattle with acaracides-chemicals which kill ticks. This approach has several short comings. For example resistance to the chemicals arises in the tick population and new classes of chemicals must be introduced frequently. The chemicals have little residual effect so cattle must be treated frequently in order to control the ticks effectively. The chemicals may have detrimental effects on the cattle, personnel and the environment. A second method for control of ticks is to breed for host resistance. Zebu breeds and Zebu cross breeds are more resistant to ticks than the highly susceptible British breeds. However Zebu crosses have behavioural problems, are less productive than pure British breeds and, even with the use of chemicals, the degree of resistance to ticks is far from ideal. Other methods of tick management such as pasture spelling and tick eradication present practical problems in most cattle producing areas throughout the world. An effective vaccine against ticks would provide a highly attractive alternative to the currently available methods of tick control.

Intermittent attempts have been made in the past to immunize animals against ticks. The majority of these studies have used tick-host systems in which strong immunity seems to develop naturally, and have usually used laboratory animals as hosts. Usually the effects observed have been some reduction in engorgement weights and egg masses of adult ticks and some decrease in the viability of those eggs although in two reports some decrease in the viability of engorging adults has been reported. Many of these studies have used antigens derived from salivary glands in order to attempt to mimic natural immunity. However, it is unlikely that a vaccine which mimics natural immunity would be of great commercial benefit due to the economic losses which still occur once natural immunity has been expressed and the deleterious effect of hypersensitivity responses to ticks.

An alternative approach is to vaccinate animals with “concealed” or “novel” antigens, “Concealed” or “novel” antigens are, in this context, components of the parasite which can be used to raise a protective immune response in animals when used (in partially or fully purified form) to vaccinate those animals, but are antigens which are not involved in naturally acquired immunity.

The successful vaccination against ticks using concealed or novel antigens has been reported. Animals were immunized with extracts of whole ticks or tick midgut. Immunization led to reductions in tick engorgement weights, feeding period, egg masses and egg viability but no significant increase in tick mortality was observed. However, the antigen fractions used in these experiments were so complex that it was not possible to identify the individual tick antigens which were responsible for the effects noted and the reasons for the effects were not investigated in detail.

Australian Patent Application No. 59707/86 claims that antigens derived from the synganglia of ticks can act as effective vaccines against tick infestation. However, there is no evidence presented that synganglia antigens can be effective alone. In this work dissected guts and synganglia were isolated, the gut cells were lysed, centrifuged and both the supernatant and pellet were used to vaccinate the same animals together, in some cases, with a cell suspension of synganglia. All cattle in the experiments reported were vaccinated with tick gut components and some received synganglia in addition.

Treating livestock and game animals to control ticks, biting flies, and similar haematophagous or noxious arthropods or other parasitic pests is essential to prevent major economic losses. Typically, these parasites pierce the skin of animals, causing damage to the hides, blood loss, and irritation, as well as transmission of deadly infectious diseases. These factors contribute to the enormous economic losses sustained by the livestock industry. Losses in livestock production (cattle, sheep, swine, and poultry) in the U.S. due to arthropod pests have been estimated at more than $3 billion. This figure does not include the cost of pest control or losses to the equine industry. Although precise figures for most countries are lacking, estimates of world-wide economic losses due to ticks and tick-borne diseases alone are in the billions of dollars.

Ticks affect approximately 800 million cattle and a similar number of sheep throughout the world. It has been estimated that the world-wide impact of tick-borne diseases of cattle at approximately $7 billion. In addition to transmission of diseases, ticks cause severe damage due to failure of cattle to achieve expected weight gains and damage to hides to be used for leather. Weight losses in cattle are estimated at 4.4 grams per Rhipicephalus appendiculatus female and 10 grams per Amblyomma hebraeum female. Estimates of losses in wildlife are unavailable; however, tick infestations of white-tailed deer (Odocoileus virginianus) in some areas are so severe that they have been reported to kill fawns. E. phagocytophila is the agent of tick-borne fever of ruminants in Europe and reported to have damaging effects on livestock in that country.

Treatment or prevention of insect and tick infestations on animals, especially animals in the wild, is a formidable task. Thus, it is not surprising that no single, universally accepted method is available for this purpose. Common practices for delivering a pesticide, e.g., an insecticide or an acaricide, to livestock include (1) direct, whole-body treatment, where the animal's body is drenched with pesticide-containing liquids; (2) systemics, where the pesticide is allowed to circulate in the host's blood; and (3) controlled-release systems, which are usually physically attached to the animal and which release pesticide continuously over a period of weeks or months.

There are disadvantageous features to all of these previously described methods. Whole body treatments involve substantial waste. In addition, for dipping or spraying, the animals must be herded and driven to, or through, the treatment area. Such procedures are both labor-intensive and stressful to the livestock. Moreover, due to the high potential for spillage, these operations pose significant environmental hazards for the surrounding area as well as health hazards for workers.

U.S. Pat. No. 5,357,902 pertains to the UF self-medicating applicator of Norval, Meltzer, Sonenshine and Burridge. This applicator contains a container for pesticide storage as opposed to the disposable, self-contained column of the subject invention which allows facile, effortless recharging with treatment material.

Lyme disease is a complex multisystem disorder caused by the tick-borne spirochete Borrelia buradorferi. This disease has three clinical stages that can overlap or occur alone: stage one—early disease, including a characteristic expanding skin lesion (erythema chronicum migrans) and constitutional flu-like symptoms; stage two—cardiac and neurological disease; and stage three—arthritis and chronic neurological syndromes. Lyme borreliosis in humans is a multisystemic disorder caused by infection with Borrelia burgdorferi. Since the first epidemiological investigations of this disease in south-central Connecticut, human cases of Lyme borreliosis have now been acquired in 43 states of the United States (Centers for Disease Control 1989, Lyme Disease—United States, 1987 and 1988. MMWR 38:668-672), five provinces of Canada, numerous countries throughout Europe and Asia), and possibly restricted foci in Australia and Africa. Between 1982-1988, reports of 13,825 cases of Lyme borreliosis were received by the Centers for Disease Control from all 50 states of the United States, (Centers for Disease Control 1989, Lyme Disease—United States, 1987 and 1988. MMWR 38:668-672), making this disease the most prevalent arthropod-borne infection in the country.

### SUMMARY OF THE INVENTION

The present invention relates to the identification and characterization of a homologue of the proinflammatory cytokine, Macrophage Migration Inhibitory Factor in the tick, Amblyomma americanum. Studying tick feeding and digestion, the inventors discovered in a cDNA library from partially-fed A. americanum ticks the first known arthropod homologue of a human cytokine, the pro-inflammatory Macrophage Migration Inhibitory Factor (MIF). The tick origin of the MIF cDNA clone was confirmed by sequencing a genomic fragment that contained the full-length tick MIF gene with two introns. Antiserum to a tick MIF-specific peptide as well as antiserum to complete tick MIF revealed the expression of tick MIF in the salivary gland and midgut tissues of A. americanum ticks. In an in vitro functional assay, recombinant tick MIF inhibited the migration of human macrophages to the same extent as recombinant human MIF.

In a first embodiment, the invention provides a substantially pure polypeptide characterized as having an amino acid sequence including amino acid residues CLSPKENKKHSAVLFEHIEKTL (SEQ ID NO:3) and a molecular weight of about 12 kD. In one aspect, the polypeptide has an amino acid sequence as set forth in SEQ ID NO:2. Also included is a substantially pure polypeptide including the contiguous amino acid sequence CLSPKENKKHSAVLFEHIEKTL (SEQ ID NO:3). The invention also includes a peptide having a sequence that is unique to tick MIF as compared with other MIF sequences. The peptide has a sequence CLSPKENKKHSAVLFEHIEKTL (SEQ ID NO:3).

The invention also includes polynucleotides encoding invention polypeptides as well as isolated polynucleotides having at least 15 continuous base pairs that hybridizes to a polynucleotide encoding a polypeptide as set forth in SEQ ID NO:2. In one aspect, the polynucleotide includes at least 15 bases in length which hybridize under moderately to highly stringent conditions to DNA encoding a polypeptide as set forth in SEQ ID NO:2.

In another embodiment, the invention provides an antibody that binds to an invention polypeptide or binds to immunoreactive fragments thereof. Such antibodies include polyclonal or monoclonal.

In another embodiment, the invention provides a method of producing tick MIF polypeptide. The method includes expressing a polynucleotide encoding an invention MIF polypeptide in a host cell; and recovering the MIF polypeptide.

In yet another embodiment, the invention provides a method of inducing an immune response to a tick polypeptide in a subject including administering to the subject a pharmaceutical composition containing an immunogenically effective amount of isolated MIF protein characterized as having an amino acid sequence comprising amino acid residues CLSPKENKKHSAVLFEHIEKTL (SEQ ID NO:3) and a molecular weight of about 12 kD. The tick may carry a pathogen such as Borrelia sp., Theileria sp., Ehrlichia sp., Babesia sp., Rickettsia sp. and tick-borne encephalitis virus, for example. The method is applicable where the subject is a human, bovine, porcine, ovine, avian, feline, canine, equine, murine, cervine, caprine, lupine, or leporidine species, for example.

In another embodiment, the invention includes a pharmaceutical composition useful for inducing an immune response to a tick in an animal including an immunogenically effective amount of an isolated MIF protein characterized as having an amino acid sequence comprising amino acid residues CLSPKENKKHSAVLFEHIEKTL (SEQ ID NO:3) and a molecular weight of about 12 kD, in a pharmaceutically acceptable carrier.

The invention also provides a method of inducing an immune response to a tick polypeptide in a subject including administering to the subject a pharmaceutical composition containing an immunogenically effective amount of isolated MIF antibody that binds to a protein characterized as having an amino acid sequence comprising amino acid residues CLSPKENKKHSAVLFEHIEKTL (SEQ ID NO:3) and a molecular weight of about 12 kD.

The invention provides a kit useful for the detection of tick MIF polynucleotide including a carrier means with at least two containers, wherein the first container contains a nucleic acid which encodes the amino acid sequence of SEQ ID NO:2 or a nucleic acid probe at least 15 bases in length that hybridizes with a nucleic acid sequence that encodes SEQ ID NO:2 or SEQ ID NO:3, and wherein a second container contains a label for detection of nucleic acid for identification of the presence of tick MIF polynucleotide.

Also included is a method for detecting antibody to tick MIF polypeptide in a sample comprising contacting the sample with tick MIF polypeptide, or fragments thereof, under conditions which allow the antibody to bind to tick MIF polypeptide and detecting the binding of the antibody to the tick MIF polypeptide, or fragments thereof.

In one embodiment is included a method for identifying a compound which binds to an invention polypeptide incubating components including the compound and the polypeptide under conditions sufficient to allow the components to interact; and measuring the binding of the compound to the polypeptide. The invention also includes a method for accelerating wound healing in a subject in need of such treatment including contacting the site of the wound with a therapeutically effective amount of a composition containing a polypeptide of the invention or functional peptide thereof.

The invention includes a method for treating a tumor or a cell proliferative disease in a subject in need of such treatment including contacting the site of the tumor or contacting the subject with a therapeutically effective amount of a composition containing a polypeptide of the invention or functional peptide thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the genomic sequence and organization for a gene for a homologue of Macrophage Migration Inhibitory Factor in Amblyomma americanum. Panel A. A genomic sequence of 4050 nucleotides is shown. The nucleotide sequence of the cDNA clone is in upper case, and the remainder of the sequence, which comprises additional 5′ flanking region, two introns, and additional 3′ flanking region is in lower case. Consensus splice sites are in bold. The start codon for open reading frame begins at position 1422. The deduced amino acid sequence of the cDNA is shown below the nucleotide sequence and in italicized single letters. Sequences of possible promoters (see text) are shaded. In the cDNA clone there was a poly-A sequence that began after position 3883 of the genomic sequence but which is not shown here. A predicted polyadenylation site occurs at positions 3853 to 3858 (double-underlined). Pyrimidine-rich and purine-rich stretches are underlined with a dashed line. Panel B. Schematic summary of the tick MIF gene detailed in panel A. Exons are represented by dark blocks and untranslated regions are shown by lines. Sizes of exons, introns and flanking sequences are shown on the figure.

FIG. 2 shows a comparison of amino acid sequences of A. americanum MIF with selected MIF proteins of vertebrates and invertebrates. Panel A. Alignment of amino acid sequences of MIF proteins of A. americanum (tick), Brugia malayi, Trichinella spiralis, G. gallus (chicken); M. musculus (mouse); H. sapiens (human), and C. elegans. Conserved prolines are indicated with inverted type, and cysteines are indicated with underlined type. The boxed sequence corresponds to the sequence of the synthetic peptide used in subsequent experiments. Panel B. Neighbor-joining CLUSTALW phylogenetic analysis of aligned MIF proteins with 1000 bootstraps. The C. elegans homologue of dopachrome tautomerase was used as the outgroup for this analysis. Bootstrap values (out of 1000) and the scale are indicated on the figure.

### DETAILED DESCRIPTION OF THE INVENTION

The present invention is based on the discovery of an arthropod polypeptide which is a homologue of Macrophage Migration Inhibitory Factor (MIF). The present invention relates to the identification and characterization of a homologue of the proinflammatory cytokine, Macrophage Migration Inhibitory Factor in the tick, Amblyomma americanum. The invention provides MIF polypeptide, polynucleotides, antibodies that bind to MIF and methods of use for inducing immunity to ticks, thereby reducing the incidence of tick-borne infections in animals. It should be understood that immunity may also be induced to other species of ticks, including Haemaphysalis spp, Otobius spp, Rhiphicephalus spp, other Ambylomma spp, Dennacentor spp, Ixodes spp and Hyalomma spp and species of Boophilus.

Examples of other species of ticks against which immunity can be induced include Otobius megnini, Rhiphicephalus appendiculatus, Dermacentor andersoni, D. variabilis, Haemaphysalis longicornis, Ambylomma variegatum and Ixodes holocyclus.

Ixodid ticks are blood feeding ectoparasites responsible for the transmission of a wide range of pathogens to humans and other vertebrates. Borrelia burgdorferi, Ehrlichia granulocytophia, Babesia microti and tick-borne encephalitis virus are prominent examples of tick-transmitted human disease. Ixodid ticks are susceptible to the host's immune system during the several days to weeks that they feed on the host's blood as parasites. During this time, the ticks consume milliliters of blood and remain embedded in the skin, where they elicit an inflammatory response. In the first several days of tick feeding the stage is set for a final burst of blood gorging before dropping from the host. The female tick balloons to more than 100 times its unfed size during the rapid feeding period. In the same period males embibe much smaller quantities of blood, move about the feeding site ensuring the mating of females and secreting proteins that may actually guard the mated females (Yang et al., 1997). The blood meal is not only necessary for the ixodid tick's sustenance; it is required development to the next life stage-larva to nymph, nymph to adult, and adult to thousands of eggs. Furthermore, transmission of a variety of pathogens also depends on a blood meal by the tick. For instance, the Lyme disease agent, Borrelia burgdorferi, migrates from the midgut to the salivary glands and then into the host only after the tick feeds on a host for a minimum time.

Tick-borne parasites include Borrelia species that cause Lyme disease, Borrelia lonestari, Borrelia anserina, Borrelia species that cause relapsing fever, Rickcttsia ricketsii, Rickettsia conori, Rickettsia sibirica, Coxiella burnetti, Theileria sp., Francisella tularensis, Ehrlichia species that cause ehrlichiosis, Cowdria species that cause heart-water disease or related disorders, Tick-borne encephalitis virus and related viruses, Colorodo Tick Fever orbivirus, Babesia species that cause babesiosis, Anaplasma species that cause anaplasmosis, viruses that causes Crimean-Congo Hemorrhagic Fever, viruses that causes Kyasanur Forest Disease.

The immune response elicited by immunization with tick MIF would result in diminished or absent transmission of one or more of these infectious agents. The action would be in inhibiting the pro-inflammatory response of the MIF, thus reducing the amount of feeding of the tick and then the number of infectious organisms or viruses that are delivered to the bite site.

The most important use of MIF as a vaccine is to reduce or eliminate transmission of one or more of these infectious agents. That would be the primary end-point for laboratory studies in experimental animals and in field trials of domestic livestock animals, companion animals, and humans. The vaccine would more likely see use in the veterinary area initially. Another end-point of tick vaccines is reduction of fecundity of the female ticks that feed on the vaccinated animal or in reduction of blood loss suffered by animals at risk of tick bites. This effect may or may not be related to interruption of transmission of infectious agents. There may be no effect on fecundity but an excellent prevention of infection transmission. This effect would be more applicable to managing a live stock herd where the overall health of the herd rather than the individual animal is uppermost.

The acquired immune responses of hosts to tick feeding and tick-secreted antigens are well documented. Trager and then many others demonstrated that repeated tick feedings on the same mammalian host lead to subsequent reductions in size of the blood meal, eggs laid, and hatched larvae (Trager 1939; Wikel 1996).

A DNA sequence coding for part or all of MIF isolated from A. americanum can be used in DNA hybridization experiments to identify related DNA sequence from other species of ticks. These latter DNA sequences can be constructed by genetic engineering techniques to obtain the expression by bacterial or eukaryote cells such as yeast, plant, insect, tick or mammalian cell lines of all or parts of the antigen from other species of ticks and provide an effective vaccine against those tick species which are responsible for morbidity or economic losses to man or morbidity and productivity losses to animals.

In accordance with the present invention an antigen derived from a tick species which antigen is capable of inducing a highly significant degree of immunity to tick challenge when used to vaccinate cattle has been purified and characterised. Further, bacterial cells which contain DNA sequences derived from a tick species have been produced and those bacterial cells which contain DNA sequences encoding portions of the tick protective antigen have been identified. The DNA sequence of the tick gene encoding that antigen has been determined, the resulting DNA sequence has been used to identify further bacterial cells containing related genes from other species of ticks. Expression of the antigen or portions of the antigen by bacteria or other microorganisms or by eukaryotic cells such as yeast, insect, tick, plant and mammalian cells grown in vitro provides a large amount of the antigen effective as an immunogen for the protection of cattle and other domestic animals against infestation by A. americanum and other tick species.

The invention also includes within its scope the epitope or the epitopes of immunogens of the invention which are responsible for the protective immune response. These epitopes may be created artificially by the synthetic production of oligopeptides which contain sequences of portions of the protective antigen which can be predicted from the results of immunochemical tests on fragments of the protective antigen produced in bacteria or generated as a result of chemical or enzymatic cleavage of the native or recombinant peptides and includes relevant epitopes from those protective antigens, oligopeptides, idiotypes and anti-idiotypes which resemble or recognise those epitopes which may have protective effects when used to actively or passively immunize animals.

In a further embodiment the invention provides methods for the purification of immunogens according to the invention and particularly protective antigens derived from ticks.

The invention also provides examples of methods which can be used to design from the amino acid sequence data, oligonucleotide sequences which are suitable for use as hybridization probes to identify nucleic acids sequences (DNA or RNA) coding for the polypeptide containing those amino acid sequences, methods for the construction of bacterial cells containing complementary DNA and genomic DNA fragments from ticks, the use of the oligonucleotides to identify bacterial cells containing complementary and genomic DNA fragments coding for that antigen, the DNA sequence of one such cDNA fragment, methods by which recombinant DNA technology can be used to produce bacterial or eukaryote cells which synthesize the protein or parts of that protein and methods for culturing those cells and for purification of tick MIF antigen or fragments thereof to be incorporated into effective vaccines against ticks.

In a preferred model, the mechanism of action of the vaccine is one in which an immune response is generated in vaccinated animals which results in ticks feeding on those animals ingesting components of the host immune system such as antibodies which interact with the surface of tick gut cells and either alone, or together with other factors in the host blood such as components of complement result in damage occuring such as lysis of the tick gut cells which in turn results in the ticks becoming unable to effectively digest blood, the tick gut becoming permeable to host blood components, to such an extent that host blood components such as albumin, haemoglobin, immunoglobulin and blood cells can be identified in the haemoloymph of the ticks and the ticks appear red in colour. This gut damage in turn results in the death of the majority of the ticks feeding on vaccinated animals before they reach engorgement stage and those few which may survive are so badly damaged that their engorgement weight is decreased and/or reproductive capacity is impaired.

The invention also relates to antibodies generated against epitopes on the antigens according to the invention (so called idiotype antibodies) and to antibodies generated against the variable region of those first antibodies, (so called anti-idiotype antibodies) which mimic the protective epitopes on the antigen and may be used as effective vaccines in either passive protection of animals (idiotypes) or active immunization of animals (anti-idiotypes) and thereby result in effective protection.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

The Tick MIF polypeptides of the present invention may be employed as an anti-tumor agent or in treating cell-proliferative disorders. Activated macrophages alone or in combination with specific anti-tumor monoclonal antibodies have considerable tumoricidal capacity. Similarly, the ability of Tick MIF to promote macrophage-mediated killing of certain pathogens indicates the employment of this molecule in treating various infections, including tuberculosis, Hunsen disease and Candida.

In addition, the ability of Tick MIF to prevent the migration of macrophages may be exploited in a therapeutic agent for treating wounds. Local application of Tick MIF at the site of injury may result in increased numbers of activated macrophages concentrated within the wound, thereby increasing the rate of healing of the wound.

In addition, Tick MIF may be employed to stimulate the immune system to increase the immunity generated against specific vaccines. MIF proteins have the ability to enhance macrophages to present antigens to T cells. Therefore, Tick MIF may be emplyed to potentiate the immune response to different antigens. This is extremely important in cases such as AIDS or AIDS related complex.

Exemplary MIF polypeptide is set forth in SEQ ID NO:2 or a tick-specific MIF peptide by SEQ ID NO:3, which includes conservative variants thereof. The terms “conservative variation” and “substantially similar” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The terms “conservative variation” and “substantially similar” also include the use of a substituted amino acid in place of an unsubstituted parent amino acid provided that antibodies raised to the substituted polypeptide also immunoreact with the unsubstituted polypeptide.

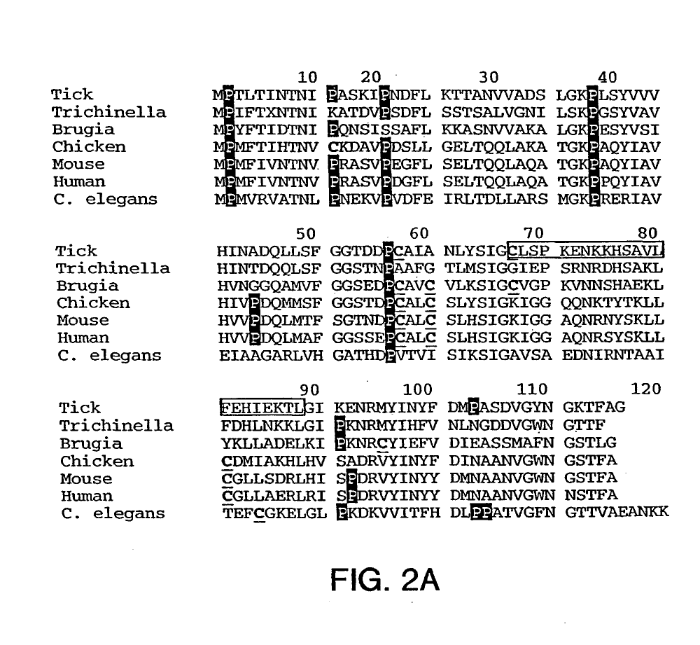

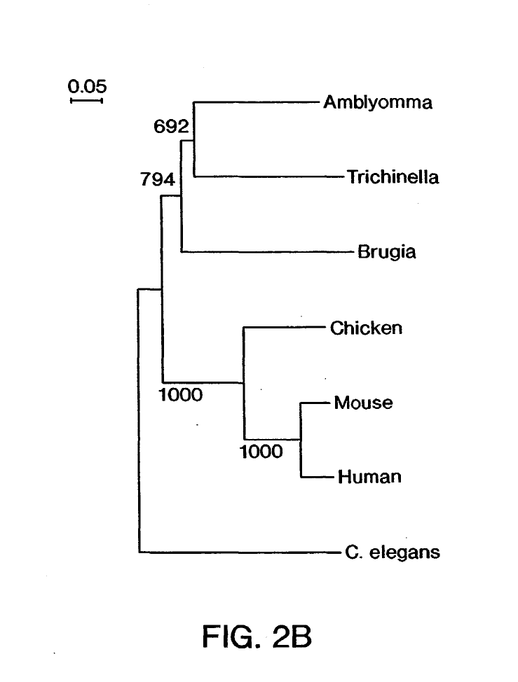

In one embodiment of the invention, the polypeptide is identical with or homologous to an MIF polypeptide, such as a tick MIF represented by SEQ ID NO:2. For instance, the MIF polypeptide preferably has an amino acid sequence at least 60% homologous to a polypeptide represented by SEQ ID Nos:2, though polypeptides with higher sequence homologies of, for example, 70%, 80%, 90% or 95% are also included herein. The MIF polypeptides can comprise a full length protein, such as represented in the sequence listings, or it can comprise a fragment of, for instance, at least 5, 10, 20, 50, 100, 150 or 200 amino acids in length. An exemplary peptide of the tick MIF of the invention includes amino acid residues 67-88 as shown in FIG. 2A (SEQ ID NO:3). SEQ ID NO:3 (encoded by SEQ ID NO:4) may be a desirable peptide for stimulating an immune response in a host, since this peptide is unique to the tick.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. The term “nucleic acid sequence encoding an MIF polypeptide” may thus refer to one or more genes within a particular individual. Moreover, individual organisms may bear different nucleotide sequences, called alleles, which code for substantially the same polypeptide. Such allelic differences may or may not result in differences in amino acid sequence of the encoded polypeptide yet still encode a protein with the same biological activity.

“Homology” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40 percent identity, though preferably less than 25 percent identity, with one of the MIF sequences of the present invention.

The term “isolated” as also used herein with respect to nucleic acids, such as DNA or RNA, refers to molecules separated from other DNAs, or RNAs, respectively, that are present in the natural source of the macromolecule. For example, an isolated nucleic acid encoding one of the subject MIF polypeptides preferably includes no more than 10 kilobases (kb) of nucleic acid sequence which naturally immediately flanks the MIF gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state:

An exemplary polynucleotide encoding MIF protein is set forth in SEQ ID NO:1. The term “polynucleotide”, “nucleic acid”, “nucleic acid sequence”, or “nucleic acid molecule” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA or genomic DNA) independent of other sequences. It also includes genomic DNA which refers to a contiguous sequence of nucleotide that includes one or more protein coding regions, introns, upstream and downstream regulatory sequences, i.e., non-coding 5′- and 3′-regulatory sequences. Thus, the term “polynucleotide encoding a polypeptide” encompasses a polynucleotide which includes coding sequence for the polypeptide as well as a polynucleotide which includes additional coding and/or non-coding sequence. For example, the nucleic acid sequence set forth in SEQ ID NO:1 includes a region encoding tick MIF protein (nucleotides 1422 to 3798) as well as regions of regulatory sequences (nucleotides 1-1421 to 3799-4050). The ORF was interrupted by two introns at 1530-2176 and 2350-3752, both having consensus splice sites.

The nucleotides of the invention can be deoxyribonucleotides, ribonucleotides in which uracil (U) is present in place of thymine (T), or modified forms of either nucleotide. The nucleotides of the invention can be complementary to the deoxynucleotides or to the ribonucleotides. A polynucleotide encoding an MIF protein includes “degenerate variants”, sequences that are degenerate as a result of the genetic code. There are 20 natural amino acids, most of which are specified by more than one codon. Therefore, all degenerate nucleotide sequences are included in the invention as long as the amino acid sequence of a polypeptide encoded by the nucleotide sequence of SEQ ID NO:1 or a peptide encoded by SEQ ID NO:4 is functionally unchanged.

A nucleic acid molecule encoding an MIF protein includes sequences encoding functional MIF polypeptides as well as functional fragments thereof. As used herein, the term “functional polypeptide” refers to a polypeptide which possesses biological function or activity which is identified through a defined functional assay (e.g., ability to inhibit migration macrophages in a migration assay (see Table 1)), and which is associated with a particular biologic, morphologic, or phenotypic alteration in the cell. The term “functional fragments of MIF protein” refers to fragments of an MIF protein that retain an MIF activity, e.g., the ability to inhibit macrophage migration. Additionally, functional CMIF fragments may act as competitive inhibitors of MIF binding, for example, biologically functional fragments, for example, can vary in size from a polypeptide fragment as small as an epitope capable of binding an antibody molecule (e.g., SEQ ID NO:3) to a large polypeptide capable of participating in the characteristic induction or programming of biological changes within a cell. Nucleotide fragments of the invention have at least 15 base pairs and hybridize to a polynucleotide encoding a polypeptide as set forth in SEQ ID NO:2 or a peptide of SEQ ID NO:2.

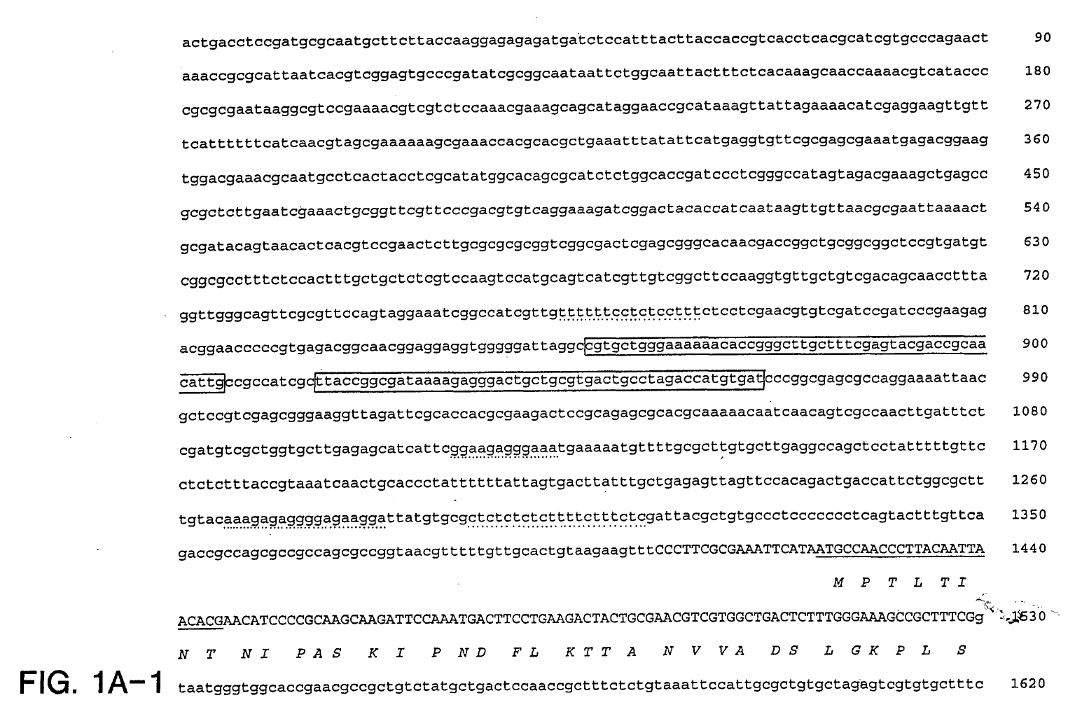

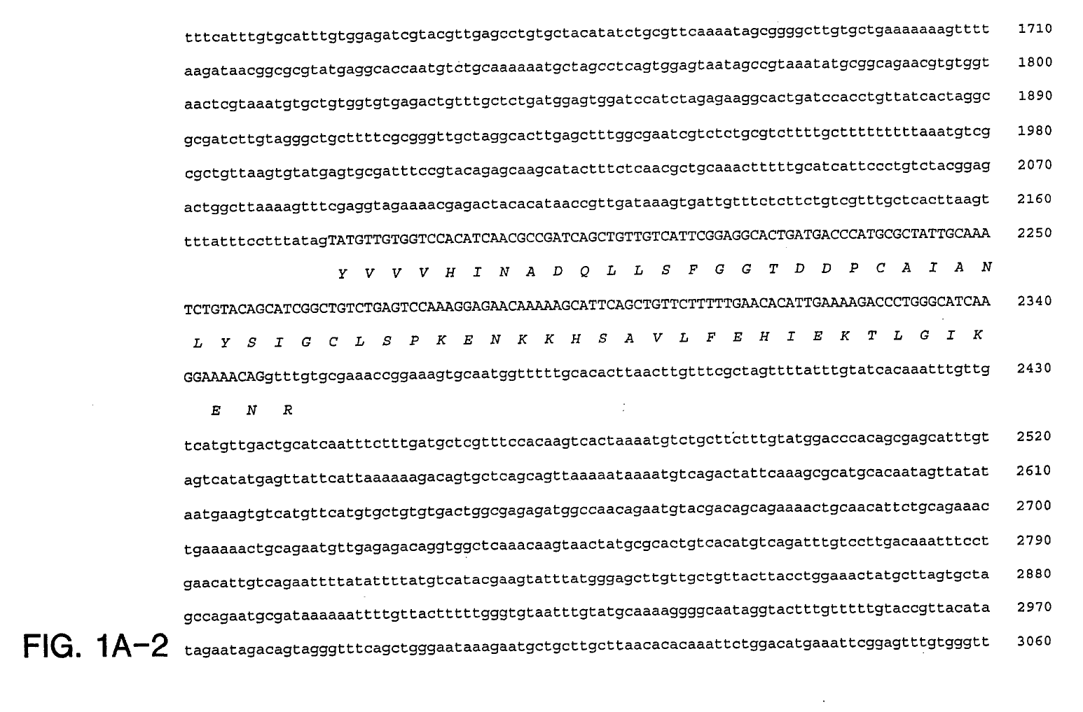

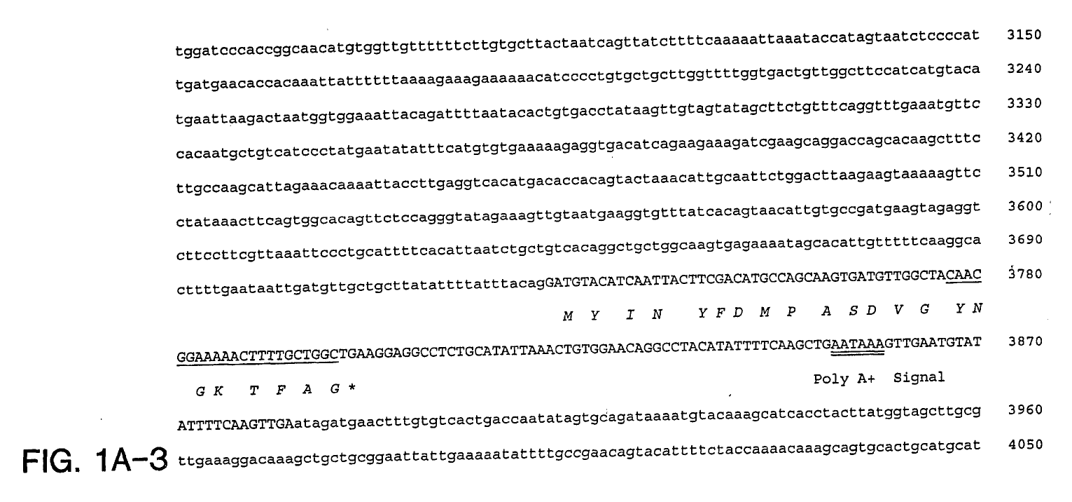

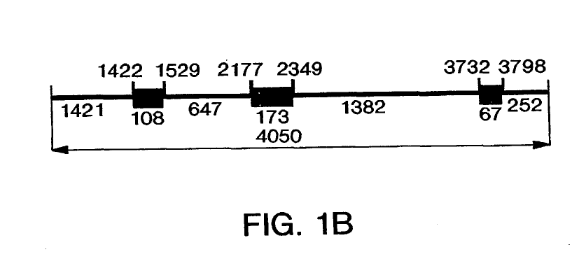

An alternative embodiment provides nucleotide fragments having at least 15 base pairs and that hybridizes to a polynucleotide encoding a polypeptide as set forth in amino acid residues 1 to 116 of FIG. 1.

Yet another embodiment of the invention provides an isolated polynucleotide, wherein the nucleotide is at least 15 base pairs in length which hybridizes under moderately to highly stringent conditions to DNA encoding a polypeptide as set forth in SEQ ID NO:2 or SEQ ID NO:3. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderately stringent conditions); and 0.1×SSC at about 68° C. (highly stringent conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Antibodies of the invention may bind to tick-specific MIF polypeptides or peptides (e.g., SEQ ID NO:3) provided by the invention to prevent normal activity of MIF proteins. Binding of antibodies to MIF proteins can interfere with, for example, the ability of a tick to feed. Binding of antibodies to MIF protein can be used to detect the presence of tick MIF in a sample.

The antibodies of the invention can be used in any subject in which it is desirable to administer in vitro or in vivo immunodiagnosis or immunotherapy. The antibodies of the invention are suited for use, for example, in immunoassays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The term “antibody” as used in this invention includes intact molecules as well as fragments thereof, such as Fab, P(ab′)2, and Fv which are capable of binding to an epitopic determinant present in an invention polypeptide. Such antibody fragments retain some ability to selectively bind with its antigen or receptor.

Methods of making these fragments are known in the art. (See for example, Harlow and Lane, Antibodies: A Laboratory Manual, Cold Spring Harbor Laboratory, New York (1988), incorporated herein by reference). Monoclonal antibodies are made from antigen containing fragments of the protein by methods well known to those skilled in the art (Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference. Briefly, monoclonal antibodies can be obtained by injecting mice with a composition comprising an antigen/ligand, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG)” in Methods In Molecular Biology, Vol. 10, pages 79-104 (Humana Press 1992).

Antibodies that bind to an invention polypeptide can be prepared using an intact polypeptide or fragments containing small peptides of interest as the immunizing antigen (e.g., SEQ ID NO:3). It may also be desirable to produce antibodies that specifically bind to the amino- or carboxyl-terminal domains of an invention polypeptide. For the preparation of polyclonal antibodies, the polypeptide or peptide used to immunize an animal is derived from translated cDNA or chemically synthesized and can be conjugated to a carrier protein, if desired. Commonly used carrier proteins which may be chemically coupled to the immunizing peptide include keyhole limpet hemocyanin (KLH), thyroglobulin, bovine serum albumin (BSA), tetanus toxoid, and the like.

Invention polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See, for example, Coligan, et al., Unit 9, Current Protocols in Immunology, Wiley Interscience, 1994, incorporated herein by reference).

The antibodies of the invention can be bound to many different carriers and used to detect the presence of an antigen comprising the polypeptides of the invention. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific antihapten antibodies.

In using the monoclonal and polyclonal antibodies of the invention for the in vivo detection of antigen, e.g., an MIF protein or peptide, the detectably labeled antibody is given a dose which is diagnostically effective. The term “diagnostically effective” means that the amount of detectably labeled antibody is administered in sufficient quantity to enable detection of the site having the antigen comprising a polypeptide of the invention for which the antibodies are specific.

The concentration of detectably labeled antibody which is administered should be sufficient such that the binding to those cells having the polypeptide is detectable compared to the background. Further, it is desirable that the detectably labeled antibody be rapidly cleared from the circulatory system in order to give the best target-to-background signal ratio.

As a rule, the dosage of detectably labeled antibody for in vivo treatment or diagnosis will vary depending on such factors as age, sex, and extent of disease of the individual. Such dosages may vary, for example, depending on whether multiple injections are given, antigenic burden, and other factors known to those of skill in the art.

A polynucleotide agent can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, L. Bioenerg. Biomemb. 25:37-42, 1993; Kirshenbaum et al., J. Clin. Invest. 92:381-387, 1993; each of which is incorporated herein by reference).

AN MIF polynucleotide of the invention can be inserted into a vector, which can be a cloning vector or a recombinant expression vector. The term “expression vector” refers to a plasmid, virus or other vehicle known in the art that has been manipulated by insertion or incorporation of a polynucleotide, particularly, with respect to the present invention, a polynucleotide encoding all or a peptide portion of an MIF protein. Such expression vectors contain a promoter sequence, which facilitates the efficient transcription of the inserted genetic sequence of the host. The expression vector generally contains an origin of replication, a promoter, as well as specific genes which allow phenotypic selection of the transformed cells. Vectors suitable for use in the present invention include, but are not limited to, the T7-based expression vector for expression in bacteria (Rosenberg, et al., Gene 56:125, 1987), the pMSXND expression vector for expression in mammalian cells (Lee and Nathans, J. Biol. Chem. 263:3521, 1988) and baculovirus-derived vectors for expression in insect cells. The DNA segment can be present in the vector operably linked to regulatory elements, for example, a promoter, which can be a T7 promoter, metallothionein I promoter, polyhedrin promoter, or other promoter as desired, particularly tissue specific promoters or inducible promoters.

Viral expression vectors can be particularly useful for introducing a polynucleotide useful in a method of the invention into a cell, particularly a cell in a subject. Viral vectors provide the advantage that they can infect host cells with relatively high efficiency and can infect specific cell types. For example, a polynucleotide encoding an MIF protein or functional peptide portion thereof can be cloned into a baculovirus vector, which then can be used to infect an insect host cell, thereby providing a means to produce large amounts of the encoded protein or peptide portion. The viral vector also can be derived from a virus that infects cells of an organism of interest, for example, vertebrate host cells such as mammalian, avian or piscine host cells. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpes virus vectors, vaccinia virus vectors, and the like (see Miller and Rosman, BioTechniques 7:980-990, 1992; Anderson et al., Nature 392:25-30 Suppl., 1998; Venna and Somia, Nature 389:239-242, 1997; Wilson, New Engl. J. Med. 334:1185-1187 (1996), each of which is incorporated herein by reference).

When retroviruses, for example, are used for gene transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

A polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

Introduction of a polynucleotide into a cell by infection with a viral vector is particularly advantageous in that it can efficiently introduce the nucleic acid molecule into a cell ex vivo or in vivo (see, for example, U.S. Pat. No. 5,399,346, which is incorporated herein by reference). Moreover, viruses are very specialized and can be selected as vectors based on an ability to infect and propagate in one or a few specific cell types. Thus, their natural specificity can be used to target the nucleic acid molecule contained in the vector to specific cell types. As such, a vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events.

A polynucleotide sequence encoding an MIF protein can be expressed in either prokaryotes or eukaryotes. Hosts can include microbial, yeast, insect and mammalian organisms. Methods of expressing polynucleotides having eukaryotic or viral sequences in prokaryotes are well known in the art, as are biologically functional viral and plasmid DNA vectors capable of expression and replication in a host. Methods for constructing an expression vector containing a polynucleotide of the invention are well known, as are factors to be considered in selecting transcriptional or translational control signals, including, for example, whether the polynucleotide is to be expressed preferentially in a particular cell type or under particular conditions (see, for example, Sambrook et al., supra, 1989).

A variety of host cell/expression vector systems can be utilized to express an MIF polypeptide coding sequence, including, but not limited to, microorganisms such as bacteria transformed with recombinant bacteriophage DNA, plasmid DNA or cosmid DNA expression vectors; yeast cells transformed with recombinant yeast expression vectors; plant cell systems infected with recombinant virus expression vectors such as a cauliflower mosaic virus or tobacco mosaic virus, or transformed with recombinant plasmid expression vector such as a Ti plasmid; insect cells infected with recombinant virus expression vectors such as a baculovirus; animal cell systems infected with recombinant virus expression vectors such as a retrovirus, adenovirus or vaccinia virus vector; and transformed animal cell systems genetically engineered for stable expression. Where the expressed MIF protein is post-translationally modified, for example, by glycosylation, it can be particularly advantageous to select a host cell/expression vector system that can effect the desired modification, for example, a mammalian host cell/expression vector system.

Depending on the host cell/vector system utilized, any of a number of suitable transcription and translation elements, including constitutive and inducible promoters, transcription enhancer elements, transcription terminators, and the like can be used in the expression vector (Bitter et al., Meth. Enzymol. 153:516-544, 1987). For example, when cloning in bacterial systems, inducible promoters such as pL of bacteriophage λ, plac, ptrp, ptac (ptrp-lac hybrid promoter) and the like can be used. When cloning in mammalian cell systems, promoters derived from the genome of mammalian cells, for example, a human or mouse metallothionein promoter, or from mammalian viruses, for example, a retrovirus long terminal repeat, an adenovirus late promoter or a vaccinia virus 7.5K promoter, can be used. Promoters produced by recombinant DNA or synthetic techniques can also be used to provide for transcription of the inserted GDF receptors coding sequence.

In yeast cells, a number of vectors containing constitutive or inducible promoters can be used (see Ausubel et al., supra, 1987, see chapter 13; Grant et al., Meth. Enzymol. 153:516-544, 1987; Glover, DNA Cloning Vol. 1 (IRL Press, 1986), see chapter 3; Bitter, Meth. Enzymol. 152:673-684, 1987; see, also, The Molecular Biology of the Yeast Saccharomyces (Eds., Strathem et al., Cold Spring Harbor Laboratory Press, 1982), Vols. 1 and 11). A constitutive yeast promoter such as ADH or LEU2 or an inducible promoter such as GAL can be used (Rothstein, DNA Cloning Vol. II (supra, 1986), chapter 3). Alternatively, vectors can be used which promote integration of foreign DNA sequences into the yeast chromosome.

Eukaryotic systems, particularly mammalian expression systems, allow for proper post-translational modifications of expressed mammalian proteins. Eukaryotic cells which possess the cellular machinery for proper processing of the primary transcript, glycosylation, phosphorylation, and advantageously, plasma membrane insertion of the gene product can be used as host cells for the expression of an MIF protein, or functional peptide portion thereof.

Mammalian cell systems which utilize recombinant viruses or viral elements to direct expression can be engineered. For example, when using adenovirus expression vectors, the MIF polypeptide coding sequence can be ligated to an adenovirus transcription/translation control complex, e.g., the late promoter and tripartite leader sequence. Alternatively, the vaccinia virus 7.5K promoter can be used (Macket et al., Proc. Natl. Acad. Sci., USA 79:7415-7419, 1982; Mackett et al., J. Virol. 49:857-864, 1984; Panicali et al., Proc. Natl. Acad. Sci., USA 79:4927-4931, 1982). Particularly useful are bovine papilloma virus vectors, which can replicate as extrachromosomal elements (Sarver et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host cell chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of the MIF protein gene in host cells (Cone and Mulligan, Proc. Natl. Acad. Sci., USA 81:6349-6353, 1984). High level expression can also be achieved using inducible promoters, including, but not limited to, the metallothionein IIA promoter and heat shock promoters.

For long term, high yield production of recombinant proteins, stable expression is preferred. Rather than using expression vectors which contain viral origins of replication, host cells can be transformed with MIF protein cDNA controlled by appropriate expression control elements such as promoter, enhancer, sequences, transcription terminators, and polyadenylation sites, and a selectable marker. The selectable marker in the recombinant plasmid can confer resistance to the selection, and allows cells to stably integrate the plasmid into their chromosomes and grow to form foci, which, in turn can be cloned and expanded into cell lines. For example, following the introduction of foreign DNA, engineered cells can be allowed to grow for 1 to 2 days in an enriched media, and then are switched to a selective media. A number of selection systems can be used, including, but not limited to, the herpes simplex virus thymidine kinase (Wigler et al., Cell 11:223, 1977), hypoxanthine-guanine phosphoribosyltransferase (Szybalska and Szybalski, Proc. Natl. Acad. Sci., USA 48:2026, 1982), and adenine phosphoribosyltransferase (Lowy, et al., Cell 22:817, 1980) genes can be employed in tk, hgprt or aprt cells respectively. Also, antimetabolite resistance can be used as the basis of selection for dhfr, which confers resistance to methotrexate (Wigler, et al., Proc. Natl. Acad. Sci. USA 77:3567, 1980; O'Hare et al., Proc. Natl. Acad. Sci., USA 78: 1527, 1981); gpt, which confers resistance to mycophenolic acid (Mulligan and Berg, Proc. Natl. Acad. Sci., USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984) genes. Additional selectable genes, including trpB, which allows cells to utilize indole in place of tryptophan; hisD, which allows cells to utilize histinol in place of histidine (Hartman and Mulligan, Proc. Natl. Acad. Sci., USA 85:8047, 1988); and ODC (ornithine decarboxylase) which confers resistance to the ornithine decarboxylase inhibitor, 2-(difluoromethyl)-DL-ornithine, DFMO (McConlogue, Curr. Comm. Mol. Biol. (Cold Spring Harbor Laboratory Press, 1987), also have been described.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be cotransformed with DNA sequences encoding the MIF proteins of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Gluzman, Eukaryotic Viral Vectors (Cold Spring Harbor Laboratory Press, 1982)).

The invention provides a method for producing a polypeptide encoded by the nucleotide sequence of SEQ ID NO:1 or SEQ ID NO:4 or fragments thereof, including culturing the host cell under conditions suitable for the expression of the polypeptide and recovering the polypeptide from the host cell culture.

An MIF polypeptide or a fragment thereof, can be encoded by a recombinant or non-recombinant nucleic acid molecule and expressed in a cell. Preparation of an MIF polypeptide by recombinant methods provides several advantages. In particular, the nucleic acid sequence encoding the MIF polypeptide can include additional nucleotide sequences encoding, for example, peptides useful for recovering the MIF polypeptide from the host cell. An MIF polypeptide can be recovered using well known methods, including, for example, precipitation, gel filtration, ion exchange, reverse-phase, or affinity chromatography (see, for example, Deutscher et al., Guide to Protein Purification in Meth. Enzymol., Vol. 182, (Academic Press, 1990)). Such methods also can be used to purify a fragment of an MIF polypeptide, for example, a particular binding sequence, from a cell in which it is naturally expressed.

A recombinant nucleic acid molecule encoding an MIF polypeptide or a fragment thereof can include, for example, a protease site, which can facilitate cleavage of the MIF polypeptide from a non-MIF polypeptide sequence, for example, a tag peptide, secretory peptide, or the like. As such, the recombinant nucleic acid molecule also can encode a tag peptide such as a polyhistidine sequence, a FLAG peptide (Hopp et al., Biotechnology 6:1204 (1988)), a glutathione S-transferase polypeptide or the like, which can be bound by divalent metal ions, a specific antibody (U.S. Pat. No. 5,011,912), or glutathione, respectively, thus facilitating recovery and purification of the MIF polypeptide comprising the peptide tag. Such tag peptides also can facilitate identification of the MIF polypeptide through stages of synthesis, chemical or enzymatic modification, linkage, or the like. Methods for purifying polypeptides comprising such tags are well known in the art and the reagents for performing such methods are commercially available.

A nucleic acid molecule encoding an MIF polypeptide can be engineered to contain one or more restriction endonuclease recognition and cleavage sites, which can facilitate, for example, substitution of an element of the MIF polypeptide such as the selective recognition domain or, where present, a spacer element. As such, related MIF polypeptides can be prepared, each having a similar activity, but having specificity for different function-forming contexts. A restriction endonuclease site also can be engineered into (or out of) the sequence coding a peptide portion of the MIF polypeptide, and can, but need not change one or more amino acids encoded by the particular sequence. Such a site can provide a simple means to identify the nucleic acid sequence, based on cleavage (or lack of cleavage) following contact with the relevant restriction endonuclease, and, where introduction of the site changes an amino acid, can further provide advantages based on the substitution.

Another embodiment of the invention provides a computer readable medium having store thereon a nucleic acid sequence of SEQ ID NO:1 or SEQ ID NO:4, and sequences substantially identical thereto, or a polypeptide sequence of SEQ ID NO:2 or SEQ ID NO:3, and sequences substantially identical thereto.

A further embodiment of the invention provides a computer system comprising a processor and a data storage device wherein said date storage device has stored thereon a nucleic acid sequence selected from the group consisting of SEQ ID NO:1, SEQ ID NO:3, and sequences substantially identical thereto, or a polypeptide sequence selected from the group consisting of SEQ ID NO:2, SEQ ID NO:4, and sequences substantially identical thereto. The computer system, additionally can contain a sequence comparison algorithm and a data storage device having at least one reference sequence stored on it. The sequence comparison algorithm comprises a computer program which indicates polymorphisms. The term “polymorphism”, as used herein, refers to the existence of multiple alleles at a single locus. Polymorphism can be are several types including, for example, those that change DNA sequence but do not change protein sequence, those that change protein sequence without changing function, those that create proteins with a different activity, and those that create proteins that are non-functional.

Homology or identity is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (see J. Roach, at the uniform resource locator (url) weber.u.Washington.edu/˜roach/human_genome_progress 2.html) (Gibbs, 1995). At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Several databases containing genomic information annotated with some functional information are maintained by different organization, and are accessible via the internet, for example, http://wwwtigr.org/tdb; http://www.genctics.wisc.edu; http://genome-www.stanford.edu/˜ball; http://hiv-web.lanl.gov; http://www.ncbi.nlm.nih.gov; http://www.ebi.ac.uk; http://Pasteur.fr/other/biology; and http://www.genome.wi.mit.edu.

One example of a useful algorithm is BLAST and BLAST 2.0 algorithms, which are described in Altschul et al., Nuc. Acids Res. 25:3389-3402, 1977, and Altschul et al., J. Mol. Biol. 215:403-410, 1990, respectively. Software for performing BLAST analyses is publicly available through the National Center for Biotechnology Information (http://www.ncbi.nhn.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectations (E) of 10, and the BLOSUM62 scoring matrix (see Henikoff & Henikoff, Proc. Natl. Acad. Sci. USA 89:10915, 1989) aligmnents (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

In one embodiment, protein and nucleic acid sequence homologies are evaluated using the Basic Local Alignment Search Tool (“BLAST”) In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST programs are accessible through the U.S. National Library of Medicine site on the world wide web, for example.

The parameters used with the above algorithms may be adapted depending on the sequence length and degree of homology studied. In some embodiments, the parameters may be the default parameters used by the algorithms in the absence of instructions from the user.

Various methods of amplifying target sequences can be used to prepare DNA encoding a polynucleotide or nucleotide fragment according to the sequence set forth in SEQ ID NO:1 or 4. Polymerase chain reaction (PCR) technology is used to amplify such nucleic acid sequences directly from mRNA, from cDNA, from genomic libaries or cDNA libraries. Isolated sequences encoding a human MIF protein may also be used as templates for PCR amplification. PCR techniques require the synthesis of oligonucleotide primers complementary to the two 3′ borders of the DNA region to be amplified. The polymerase chain reaction is then performed using two primers. (see PCR Protocols: A Guide to Methods and Applications, Innis, Gelfand, Sninsky, and White, eds., Academic Press, San Diego (1990).) Primers can be selected to amplify the entire region or regions that encode the full-length sequence of interest or to amplify smaller DNA segments.

Various methods of screening and detecting nucleic acid mutations and polymorphisms are known in the art including hybridization with allele-specific oligonucleotide probes, including immobilized oligonucleotides and oligonucleotide arrays, allele-specific PCR (Newton et al. (1989) Nucl. Acids, Res. 17:2503-2516), mismatch-repair detection (Faham and Cox (1995) Genome Res. 5:474-482); restriction fragment length polymorphism detection based on allele-specific restriction endonuclease cleavage (Kan and Dozy (1978) Lancet 2: 910-912), hybridization with allele-specific oligonucleotide probes (Wallace et al. (1978) Nucl Acids Res 6: 3543-3557), including immobilized oligonucleotides (Saiki et al. (1989) Proc. Natl. Acad. Sci. U.S.A. 86: 6230-6234) or oligonucleotide arrays (Maskos and Southern (1993) Nucl Acids Res 21: 2269-2270), binding of MutS protein (Wagner et al. (1995) Nuel Acids Res 23: 3944-3948, denaturing-gradient gel electrophoresis (DGGE) (Fisher and Lerman et al. (1983) Proc. Natl. Acad. Sci. U.S.A. 80: 1579-1583), single-strand-conformation-polymorphism detection (Orita et al. (1983) Genomics 5: 874-879), RNAase cleavage at mismatched base-pairs (Myers et al. (1985) Science 230: 1242), chemical (Cotton et al (1988). Proc. Natl. Acad. Sci. U.S.A. 85: 4397-4401) or enzymatic (Youil et al. (1995) Proc. Natl. Acad. Sci. U.S.A. 92: 87-91) cleavage of heteroduplex DNA, methods based on allele specific primer extension (Syvanen et al. (1990) Genomics 8: 684-692), genetic bit analysis (GBA) (Nikiforov et al. (1994) Nucl Acids Res 22: 4167-4175), the oligonucleotide-ligation assay (OLA) (Landegren et al. (1988) Science 241: 1077), the allele-specific ligation chain reaction (LCR) (Barrany (1991) Proc. Natl. Acad. Sci. U.S.A. 88: 189-193), gap-LCR (Abravaya et al. (1995) Nucl Acids Res 23: 675-682), and radioactive and/or fluorescent DNA sequencing using standard procedures well known in the art.

In another embodiment, the invention provides a method for identifying a compound which binds to a polypeptide of SEQ ID NO:2 or a peptide of SEQ ID NO:3. The method includes incubating components comprising the compound and the polypeptide under conditions sufficient to allow the components to interact; and measuring the binding of the compound to the polypeptide. Compounds that bind to SEQ ID NO:2 or NO:3 include peptides, peptidomimetics, and antibodies, for example.

In another embodiment, the invention provides a method for identifying a compound which binds to MIF. The method includes incubating components comprising the compound and MIF under conditions sufficient to allow the components to interact and measuring the binding of the compound to MIF. Compounds that bind to MIF include peptides, peptidomimetics, polypeptides, chemical compounds and biologic agents as described above.

Incubating includes conditions which allow contact between the test compound and MIF. Contacting includes in solution and in solid phase. The test ligand(s)/compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3: 1008-1012, 1985), allele-specific oligonucleotide (ASO) probe analysis (Conner, et al., Proc. Natl. Acad. Sci. USA, 80:278, 1983), oligonucleotide ligation assays (OLAs) (Landegren, et al., Science, 241:1077, 1988), and the like. Molecular techniques for DNA analysis have been reviewed (Landegren, et al., Science, 242:229-237, 1988).

The term “incubating” includes conditions which allow contact between the test compound and the cell of interest. “Contacting” may include in solution or in solid phase.

Compounds which modulate a cellular response can include peptides, peptidomimeties, polypeptides, pharmaceuticals, chemical compounds and biological agents, for example. Antibodies, neurotropic agents, anti-epileptic compounds and combinatorial compound libraries can also be tested using the method of the invention. One class of organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

The test agent may also be a combinatorial library for screening a plurality of compounds. Compounds such as peptides identified in the method of the invention can be further cloned, sequenced, and the like, either in solution of after binding to a solid support, by any method usually applied to the isolation of a specific DNA sequence Molecular techniques for DNA analysis (Landegren et al., Science 242:229-237, 1988) and cloning have been reviewed (Sambrook et al., Molecular Cloning: a Laboratory Manual, 2nd Ed.; Cold Spring Harbor Laboratory Press, Plainview, N.Y., 1998, herein incorporated by reference).

Candidate compounds are obtained from a wide variety of sources including libraries of synthetic or natural compounds. For example, numerous means are available for random and directed synthesis of a wide variety of organic compounds and biomolecules, including expression of randomized oligonucleotides and 5 oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

A variety of other agents may be included in the screening/identification assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents and the like may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 10 h will be sufficient.

Generally, the terms “treating,” “treatment,” and the like are used herein to mean obtaining a desired pharmacologic and/or physiologic effect. The effect may be prophylactic in terms of completely or partially preventing a spirochete infection or disease (e.g., leptospirosis or Lyme disease) or sign or symptom thereof, and/or may be therapeutic in terms of a partial or complete cure for an infection or disease and/or adverse effect attributable to the infection or disease. “Treating” as used herein covers any treatment of (e.g., complete or partial), or prevention of, an infection or disease in a subject, and includes:

(a) preventing the disease from occurring in a subject that may be predisposed to the disease, but has not yet been diagnosed as having it;

(b) inhibiting the infection or disease, i.e., arresting its development; or

(c) relieving or ameliorating the infection or disease, i.e., cause regression of the infection or disease.

Thus, the invention includes various pharmaceutical compositions useful for ameliorating symptoms attributable to a tick-borne infection or, alternatively, for inducing a protective immune response to prevent transmission of a pathogen. The pharmaceutical compositions according to the invention are prepared by bringing an antibody against MIF, MIF polypeptide, a functional peptide or peptide derivative of MIF (e.g., SEQ ID NO:3), an MIF mimetic, or an MIF-binding agent according to the present invention into a form suitable for administration to a subject using carriers, excipients and additives or auxiliaries. It is envisioned that MIF can be utilized in topical preparations or the like for modulating an immune response in the skin, for example. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally (e.g., topically) or systemically. By “therapeutically effective dose” is meant the quantity of a compound according to the invention necessary to prevent, to cure or at least partially arrest the symptoms of the disease and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

In one embodiment, the invention provides a pharmaceutical composition useful for inducing an immune response to a tick in an animal comprising an immunologically effective amount of MIF in a pharmaceutically acceptable carrier. Alternatively, the MIF polypeptide of functional peptides thereof may be useful for modulating an immune response, given the effect of MIF on macrophage migration. “Administering” the pharmaceutical composition of the present invention may be accomplished by any means known to the skilled artisan. By “subject” is meant any animal, including human, bovine, porcine, ovine, avian, feline, canine, equine, murine, cervine, caprine, lupine, or leporidine species. The term “immunogenically effective amount,” as used in describing the invention, is meant to denote that amount of antigen which is necessary to induce, in an animal, the production of a protective immune response to a tick. The MIF protein of the invention is particularly useful in sensitizing the immune system of an animal such that, as one result, an immune response is produced which ameliorates the effect of a transmission of tick-borne infection.

The MIF protein can be administered parenterally by injection, rapid infusion, nasopharyngeal absorption, dermal absorption, and orally. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water.

In addition to the inert diluents, such compositions can also include adjuvants, wetting agents, and emulsifying and suspending agents. Adjuvants are substances that can be used to nonspecifically augment a specific immune response. Normally, the adjuvant and the antigen are mixed prior to presentation to the immune system, or presented separately, but into the same site of the animal being immunized. Adjuvants can be loosely divided into several groups based on their composition. These groups include oil adjuvants (for example, Freund's Complete and Incomplete), mineral salts (for example, AlK(SO₄)₂, AlNa(SO₄)₂, AlNH₄(SO₄), silica, alum, Al(OH)₃, Ca₃(PO₄)₂, kaolin, and carbon), polynucleotides (for example, poly IC and poly AU acids), and certain natural substances (for example, wax D from Mycobacterium tuberculosis, as well as substances found in Corynebacterium parvum, Bordetella pertussis, and members of the genus Brucella).

The method of the invention also includes slow release antigen delivery systems such as microencapsulation of antigens into liposomes. Such systems have been used as an approach to enhance the immunogenicity of proteins without the use of traditional adjuvants. Liposomes in the blood stream are generally taken up by the liver and spleen, and are easily phagocytosed by macrophages. Liposomes also allow co-entrapment of immunomodulatory molecules along with the antigens, so that such molecules may be delivered to the site of antigen encounter, allowing modulation of the immune system towards protective responses.

Many different techniques exist for the timing of the immunizations when a multiple immunization regimen is utilized. It is possible to use the antigenic preparation of the invention more than once to increase the levels and diversity of expression of the immune response of the immunized animal. Typically, if multiple immunizations are given, they will be spaced two to four weeks apart. Subjects in which an immune response to a tick is desirable include humans, dogs, cattle, horses, deer, mice, goats, wolves and sheep.

Generally, the dosage of MIF protein administered to a subject will vary depending on such factors as age, condition, sex and extent of disease, if any, and other variables which can be adjusted by one of ordinary skill in the art.

In addition to generating antibodies which bind to antigenic epitopes of MIF, it is further envisioned that the method of the invention can be used to induce cellular responses, particularly cytotoxic T-lymphocytes (CTLs), to antigenic epitopes of MIF. Typically, unmodified soluble proteins fail to prime major histocompatibility complex (MIC) class I-restricted CTL responses whereas particulate proteins are extremely immunogenic and have been shown to prime CTL responses in vivo. CTL epitopes and helper epitopes have been identified in proteins from many infectious pathogens. Further, these epitopes can be produced concurrently such that multiple epitopes can be delivered in a form that can prime MHC class I restricted CTL responses. An example of a system that can produce recombinant protein particles carrying one or more epitopes entails the use of the p1 protein of the retrotransposon Ty1 of Saccharomyces cerevisiae (Adams, et al., Nature, 329:68, 1987). Sequences encoding CTL epitopes can, for example, be fused to the C-terminus of p1 and the resulting Ty virus-like particles (Ty-VLPs) may be able to generate a CTL response. Thus, conserved regions of tick MIF antigens can be identified and incorporated together in a particle which enables the host immune system to mount an effective immune response against multiple tick species. Further, the method of the invention can be used to generate particles with multiple epitopes to a single protein, such as MIF, or multiple epitopes from various tick MIF proteins.

In a further embodiment, the invention provides a method of detecting a tick-associated MIF nucleic acid, protein or antibody in a subject comprising contacting a cell component containing MIF with a reagent which binds to the cell component. The cell component can be nucleic acid, such as DNA or RNA, or it can be protein. When the component is nucleic acid, the reagent is a nucleic acid probe or PCR primer. When the cell component is protein, the reagent is an antibody probe. The probes are detectably labeled, for example, with a radioisotope, a fluorescent compound, a bioluminescent compound, a chemiluminescent compound, a metal chelator or an enzyme. Those of ordinary skill in the art will know of other suitable labels for binding to the antibody, or will be able to ascertain such, using routine experimentation.

For purposes of the invention, an antibody or nucleic acid probe specific for MIF may be used to detect the presence of MIF polypeptide (using antibody); MIF antibody (using MIF polypeptide or peptide) or polynucleotide (using nucleic acid probe) in biological fluids or tissues. Any specimen containing a detectable amount of MIF antigen, antibody, or polynucleotide can be used. A preferred specimen of this invention is blood.

Another technique which may also result in greater sensitivity consists of coupling antibodies to low molecular weight haptens. These haptens can then be specifically detected by means of a second reaction. For example, it is common to use such haptens as biotin, which reacts with avidin, or dinitrophenyl, pyridoxal, and fluorescein, which can react with specific antihapten antibodies.

Alternatively, MIF polypeptide can be used to detect antibodies to MIF polypeptide in a specimen. The MIF of the invention is particularly suited for use in immunoassays in which it can be utilized in liquid phase or bound to a solid phase carrier. In addition, MIF used in these assays can be detectably labeled in various ways.

Examples of immunoassays which can utilize the MIF of the invention are competitive and noncompetitive immunoassays in either a direct or indirect format. Examples of such immunoassays are the radioimmunoassay (RIA), the sandwich (immunometric assay) and the Western blot assay. Detection of antibodies which bind to the MIF of the invention can be done utilizing immunoassays which run in either the forward, reverse, or simultaneous modes, including immunohistochemical assays on physiological samples. The concentration of MIF which is used will vary depending on the type of immunoassay and nature of the detectable label which is used. However, regardless of the type of immunoassay which is used, the concentration of MIF utilized can be readily determined by one of ordinary skill in the art using routine experimentation.

The MIF of the invention can be bound to many different carriers and used to detect the presence of antibody specifically reactive with the polypeptide. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for purposes of the invention. Those skilled in the art will know of other suitable carriers for binding MIF or will be able to ascertain such, using routine experimentation.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds.

For purposes of the invention, the antibody which binds to MIF of the invention may be present in various biological fluids and tissues. Any sample containing a detectable amount of antibodies to MIF can be used. Typically, a sample is a liquid such as urine, saliva, cerebrospinal fluid, blood, serum and the like, or a solid or semi-solid such as tissue, feces and the like. Preferably, the sample is serum or blood from the subject.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 key range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr, and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ⁵⁷Gd, ⁵⁵Mn, ¹⁶²Dy, ⁵²Cr, and ⁵⁶Fe.

In another embodiment, nucleic acid probes can be used to identify MIF nucleic acid, polypeptide, or antibodies from a specimen obtained from a subject. Examples of species from which nucleic acid sequence encoding MIF can be derived because of infection include human, swine, porcine, feline, canine, equine, murine, cervine, caprine, lupine, leporidine and bovine species. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res. 9:879, 1981).

In an embodiment of the invention, purified nucleic acid fragments containing intervening sequences or oligonucleotide sequences of 10-50 base pairs are radioactively labeled. The labeled preparations are used to probe nucleic acid from a specimen by the Southern hybridization technique. Nucleotide fragments from a specimen, before or after amplification, are separated into fragments of different molecular masses by gel electrophoresis and transferred to filters that bind nucleic acid. After exposure to the labeled probe, which will hybridize to nucleotide fragments containing target nucleic acid sequences, binding of the radioactive probe to target nucleic acid fragments is identified by autoradiography (see Genetic Engineering, 1, ed. Robert Williamson, Academic Press, (1981), 72-81). Alternatively, nucleic acid from the specimen can be bound directly to filters to which the radioactive probe selectively attaches by binding nucleic acids having the sequence of interest. Specific sequences and the degree of binding is quantitated by directly counting the radioactive emissions.

Where the target nucleic acid is not amplified, detection using an appropriate hybridization probe may be performed directly on the separated nucleic acid. In those instances where the target nucleic acid is amplified, detection with the appropriate hybridization probe would be performed after amplification.

The probes of the present invention can be used for examining the distribution of the specific fragments detected, as well as the quantitative (relative) degree of binding of the probe for determining the occurrence of specific strongly binding (hybridizing) sequences, thus indicating the likelihood for an subject having or predisposed to having increased muscle mass.

For the most part, the probe will be detectably labeled with an atom or inorganic radical, most commonly using radionuclides, but also heavy metals can be used. Conveniently, a radioactive label may be employed. Radioactive labels include ³²P, ¹²⁵I, ³H, ¹⁴C, ¹¹¹In, ^{99m}Tc, or the like. Any radioactive label may be employed which provides for an adequate signal and has sufficient half-life. Other labels include ligands, which can serve as a specific binding pair member for a labeled ligand, and the like. A wide variety of labels routinely employed in immunoassays can readily be employed in the present assay. The choice of the label will be governed by the effect of the label on the rate of hybridization and binding of the probe to mutant nucleotide sequence. It will be necessary that the label provide sufficient sensitivity to detect the amount of mutant nucleotide sequence available for hybridization. Other considerations will be ease of synthesis of the probe, readily available instrumentation, ability to automate, convenience, and the like.

The manner in which the label is bound to the probe will vary depending upon the nature of the label. For a radioactive label, a wide variety of techniques can be employed. Commonly employed is nick translation with an a ³²P-dNTP or terminal phosphate hydrolysis with alkaline phosphatase followed by labeling with radioactive ³²P employing ³²P-NTP and T4 polynucleotide kinase. Alternatively, nucleotides can be synthesized where one or more of the elements present are replaced with a radioactive isotope, e.g., hydrogen with tritium. If desired, complementary labeled strands can be used as probes to enhance the concentration of hybridized label.

Where other radionucleotide labels are involved, various linking groups can be employed. A terminal hydroxyl can be esterified, with inorganic acids, e.g., ³²P phosphate, or ¹⁴C organic acids, or else esterified to provide linking groups to the label. Alternatively, intermediate bases may be substituted with activatable linking groups that can then be linked to a label.

Enzymes of interest as reporter groups will primarily be hydrolases, particularly esterases and glycosidases, or oxidoreductases, particularly peroxidases. Fluorescent compounds include fluorescein and its derivatives, rhodamine and its derivatives, dansyl, umbelliferone, and so forth. Chemiluminescers include luciferin, and 2,3-dihydrophthalazinediones (e.g., luminol).

The probe can be employed for hybridizing to a nucleotide sequence affixed to a water insoluble porous support. Depending upon the source of the nucleic acid, the manner in which the nucleic acid is affixed to the support may vary. Those of ordinary skill in the art know, or can easily ascertain, different supports that can be used in the method of the invention.

The nucleic acid from a specimen can be cloned and then spotted or spread onto a filter to provide a plurality of individual portions (plaques). The filter is an inert porous solid support, e.g., nitrocellulose. Any cells (or phage) present in the specimen are treated to liberate their nucleic acid. The lysing and denaturation of nucleic acid, as well as the subsequent washings, can be achieved with an appropriate solution for a sufficient time to lyse the cells and denature the nucleic acid. For lysing, chemical lysing will conveniently be employed, as described previously for the lysis buffer. Other denaturation agents include elevated temperatures, organic reagents, e.g., alcohols, amides, amines, ureas, phenols and sulfoxides or certain inorganic ions, e.g., thiocyanate and perchlorate.

After denaturation, the filter is washed in an aqueous buffered solution, such as Tris, generally at a pH of about 6 to 8, usually 7. One or more washings may be involved, conveniently using the same procedure as employed for the lysing and denaturation. After the lysing, denaturing, and washes have been accomplished, the nucleic acid spotted filter is dried at an elevated temperature, generally from about 50° C. to 70° C. Under this procedure, the nucleic acid is fixed in position and can be assayed with the probe when convenient.

Pre-hybridization may be accomplished by incubating the filter with the hybridization solution without the probe at a mildly elevated temperature for a sufficient time to thoroughly wet the filter. Various hybridization solutions may be employed, comprising from about 20% to 60% volume, preferably 30%, of an inert polar organic solvent. A common hybridization solution employs about 50% formamide, about 0.5 to 1M sodium chloride, about 0.05 to 0.1M sodium citrate, about 0.05 to 0.2% sodium dodecylsulfate, and minor amounts of EDTA, ficoll (about 300-500 kD), polyvinylpyrrolidone, (about 250-500 kD) and serum albumin. Also included in the hybridization solution will generally be from about 0.5 to 5 mg/ml of sonicated denatured DNA, e.g., calf thymus of salmon sperm; and optionally from about 0.5 to 2% wt/vol glycine. Other additives may also be included, such as dextran sulfate of from about 100 to 1,000 kD and in an amount of from about 8 to 15 weight percent of the hybridization solution.

The particular hybridization technique is not essential to the invention. Other hybridization techniques are described by Gall and Pardue, (Proc. Natl. Acad. Sci. 63:378, 1969); and John, et al., (Nature, 223:582, 1969). As improvements are made in hybridization techniques they can readily be applied in the method of the invention.

The amount of labeled probe present in the hybridization solution will vary widely, depending upon the nature of the label, the amount of the labeled probe that can reasonably bind to the filter, and the stringency of the hybridization. Generally, substantial excess over stoichiometric concentrations of the probe will be employed to enhance the rate of binding of the probe to the fixed target nucleic acid.

In nucleic acid hybridization reactions, the conditions used to achieve a particular level of stringency will vary, depending on the nature of the nucleic acids being hybridized. For example, the length, degree of complementarity, nucleotide sequence compound (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

After the filter has been contacted with a hybridization solution at a moderate temperature for a period of time sufficient to allow hybridization to occur, the filter is then introduced into a second solution having analogous concentrations of sodium chloride, sodium citrate and sodium dodecylsulfate as provided in the hybridization solution. The time the filter is maintained in the second solution may vary from five minutes to three hours or more. The second solution determines the stringency, dissolving cross duplexes and short complementary sequences. After rinsing the filter at room temperature with dilute sodium citrate-sodium chloride solution, the filter may now be assayed for the presence of duplexes in accordance with the nature of the label. Where the label is radioactive, the filter is dried and exposed to X-ray film.

The label may also comprise a fluorescent moiety that can then be probed with a specific fluorescent antibody. Horseradish peroxidase enzyme can be conjugated to the antibody to catalyze a chemiluminescent reaction. Production of light can then be seen on rapid exposure to film.

The materials for use in the method of the invention are ideally suited for the preparation of a kit. Such a kit may comprise a carrier means being compartmentalized to receive one or more container means such as vials, tubes, and the like, each of the container means comprising one of the separate elements to be used in the method. For example, one of the container means may comprise an MIF binding reagent, such as an antibody or nucleic acid. A second container may further comprise MIF polypeptide. The constituents may be present in liquid or lyophilized form, as desired.

One of the container means may comprise a probe which is or can be detectably labeled. Such probe may be an antibody or nucleotide specific for a target protein, or fragments thereof, or a target nucleic acid, or fragment thereof, respectively, wherein the target is indicative, or correlates with, the presence of MIF. For example, oligonucleotide probes of the present invention can be included in a kit and used for examining the presence of MIF nucleic acid, as well as the quantitative (relative) degree of binding of the probe for determining the occurrence of specific strongly binding (hybridizing) sequences, thus indicating the likelihood for an subject having a tick injection.

The kit may also contain a container comprising a reporter-means, such as a biotin-binding protein, such as avidin or streptavidin, bound to a reporter molecule, such as an enzymatic, fluorescent, or radionucleotide label to identify the detectably labeled oligonucleotide probe.

Where the kit utilizes nucleic acid hybridization to detect the target nucleic acid, the kit may also have containers containing nucleotide(s) for amplification of the target nucleic acid sequence. When it is desirable to amplify the target nucleic acid sequence, such as MIF nucleic acid sequences, this can be accomplished using oligonucleotide(s) that are primers for amplification. These oligonucleotide primers are based upon identification of the flanking regions contiguous with the target nucleotide sequence.

The kit may also include a container containing antibodies which bind to a target protein, or fragments thereof. Thus, it is envisioned that antibodies which bind to MIF, or fragments thereof (e.g., SEQ ID NO:3), can be included in a kit.

Without further elaboration, it is believed that one skilled in the art can, using the preceding description, utilize the present invention to its fullest extent. The following examples are to be considered illustrative and thus are not limiting of the remainder of the disclosure in any way whatsoever.

### EXAMPLE 1

Identification of a tick MIF. Using midgut mRNA from A. americanum females ticks that fed for three days, we produced a cDNA expression library in bacteriophage lambda. Random plaques were subcloned into a bacterial plasmid vector, and sequenced. The sequences were compared with other sequences in nucleotide and protein databases. Several of the clones had inserts that were identical to either the 12S or 16S mitochondrial rRNA genes of A. americanum. Other sequences included the cytochrome oxidases of tick mitochondria, and these will be presented in detail elsewhere. Sequences identical or similar to rabbit or other mammalian genes were not observed among 129 cDNA clones that were randomly sequenced.

The cDNA clone that is the subject of this report had a 466 bp insert with an open reading frame (ORF) of 348 nucleotides. With primers representing 5′ and 3′ ends of the ORF, we amplified a 2377 fragment from genomic DNA. 5′ and 3′ flanking regions of 1420 and 252 nucleotides, respectively, were obtained using a genome walking procedure. The entire sequence of 4050 nucleotides is shown in panel A of FIG. 1. A schematic summary of the genomic sequence is shown in panel B. Sequence corresponding to the cDNA clone is shown in upper case, and additional sequence from the genomic clones is shown in lower case. The open reading frame (ORF) of the cDNA clone was 348 nucleotides and encoded a protein of 116 amino acids. The ORF began at position 1422 of the genomic sequence and was interrupted by two introns, the first from positions 1530 to 2176, and the second from position 2350 to 3752. These introns had consensus splice. sites. The translated sequences of the introns were not discernibly homologous to known proteins by the BLASTX algorithm. The cDNA and genomic sequences for A. americanum MIF have been assigned Genbank accession numbers AF126688 and AF289543.

The cDNA clone contained 87 nucleotides 3′ to the stop codon. This contained a possible polyadenylation site (AATAAA) at positions 3853 to 3858. In the cDNA sequence a poly-A+ sequence began at position 3883 of the genomic sequence. Upstream of the start codon in the cDNA was an additional 18 nucleotides of sequence. The 5′ flanking region also contained stretches of pyrimidine-rich and purine-rich sequences. Using the Neural Network Promoter Prediction algorithm (http://www.fruitfly.org/cgi-bin/seq_tools/promoter.p1), we identified two promoter regions in the upstream sequence that had correlation coefficients of 0.98 and 0.95, respectively, where a consensus eukaryotic promoter would have a value of 1.0 (panel A of FIG. 1).

Protein sequence comparison. The deduced amino acid sequence of the ORF was highly similar to the MIF proteins of the vertebrates and parasitic nematodes. FIG. 2A shows an alignment of the deduced tick sequence with MIF proteins of human (accession number M95775), mouse (L07607), chicken (M95776), and the nematodes Brugia malayi (U88035) and Trichinella spiralis (AJ012740). Highlighted prolines emphasize the conservation seen in the groups shown (FIG. 2A). The boxed region contains a region of amino acid sequence unique to the tick sequence and a synthetic peptide was produced using this sequence (FIG. 2A). The alignment also includes a homologous, hypothetical protein of unknown function that was discovered in the genome sequence of the free-living nematode C. elegans (Z78012). The deduced protein of A. americanum was 53% identical to the T. spiralis protein, 48% identical to the B. malayi protein, 40% identical to human MIF, and 28% identical to the hypothetical protein of C. elegans. Like the parasitic helminths B. malayi and T. spiralis and the mammalian MIF, the tick MIF has proline-cysteine-alanine at positions 56-58; the C. elegans homologue has a proline-valine-threonine. Neighbor-joining tree of the amino acid sequences shows that the divisions between tick and other invertebrate proteins are deep (FIG. 2B). Neither the Saccharomyces cervisiae genome nor the D. melanogaster genome had a discernible homologue to MIF by BLASTP or TBLASTN (http://www.ncbi.nln.nih.gov/BLAST).

Expression and purification of recombinant tick MIF. Having identified an orthologue of MIF proteins in a tick cDNA library, we next expressed the recombinant tick MIF as a non-fusion protein in E. coli. The recombinant protein was purified from the bacterial lysate by anion exchange column and then reversed phase liquid chromatography (FIG. 3). The gradient was 15 to 35% acetonitrile over the first 10 minutes and then 35% to 50% acetonitrile over the next 45 minutes. Two major peaks of protein eluted between 43 and 45% acetonitrile from the column. By mass spectroscopy, peak 1 was 12,472 daltons and peak 2 was 12,633 daltons. The expected molecular weight for tick MIF from the deduced amino acid sequence was 12, 607 daltons. The peak 2 sample was blocked to N-terminal sequencing, while sequencing of a sample of peak 1 revealed the N-terminal sequence PTLTINT. The blockage of the peak 2 sample and its larger size were consistent with the presence of N-terminal formylated methionine. Cleavage of the amino-terminal methionine by a specific peptidase in E. coli likely produced peak 1, tick MIF (Ben-Bassat et al. 1987). Peak 1 was used for all of the subsequent analyses, because of the reported importance of an N-terminal proline for MIF function (Swope et al. 1998).

Specificity of polyclonal antisera to tick MIF. Polyclonal antisera was produced to the recombinant tick MIF and to a peptide of the tick MIF conjugated to keyhole limpet hemocyanin (KLH). The peptide represented residues 67 to 88 of the deduced protein and included a region of the protein that was more variable in sequence between the different MIF proteins (FIG. 2A). Control sera were from non-immunized rabbits or rabbits immunized with KLH alone. Antibodies in both the anti-tick MIF serum and the anti-peptide serum bound to recombinant tick MIF by Western blot analysis. Neither the anti-tick MIF nor the anti-peptide antibodies detected human or mouse MIF proteins by Western blot analysis. Antisera to mouse MIF did not bind to recombinant tick MIF either by Western blot or ELISA. In the sandwich ELISA for mammalian MIF, there was no detectable binding of antibody raised against mammalian MIF to tick MIF or to the anti-MIF peptide antibodies to mouse MIF.

Tissue expression of tick MIF. Having demonstrated the specificity of anti-peptide and anti-tick MIF antisera, we next used these reagents for detecting tick MIF in tissues. By Western blot analysis there was an immunoreactive protein of the same apparent size as recombinant tick MIF in the salivary glands and midgut of 3-day fed A. americanum females but not in Drosophila tissues. MIF was also detected in unfed tick salivary gland and midgut tissues, but not unconcentrated tick hemolymph. With equal amounts of protein loaded per gel lane, it is clear that the salivary glands of unfed females had less MIF than glands from partially fed females. The antiserum to KLH alone and normal rabbit serum did not detectably bind to proteins in any of these tissues. Using dilutions of the tissue extracts, we detected tissue MIF down to 2.5 mg of protein loaded per gel lane for both salivary glands and to 1.25 mg for midgut.

Biological activity of the tick MIF. The activity of purified recombinant tick MIF was assessed in a macrophage migration assay and compared with the activity of purified recombinant human MIF (Table 1). The chemokine Monocyte Chemotactic Protein-1 stimulated macrophage migration to approximately twice that of the buffer control. Tick MIF was equivalent to human MIF in its ability to inhibit the migration of macrophages. At a concentration of 100 ng/ml, tick and human MIFs inhibited macrophage migration by 53% and 57%, respectively, in comparison to the medium control. There was comparably less inhibition for both preparations at 10 ng/ml. Mammalian MIF also exhibits a dopachrome tautomerase activity (Rosengren et al. 1997). At 0.1 to 1 mg/100 ml, tick MIF had dopachrome tautomerase activity above that of negative control samples, but approximately 10-fold less than that of recombinant human MIF in equivalent concentrations.

Many of the molecules that a tick secretes into the feeding lesion facilitate ingestion of blood (Nuttall 1998; Sauer et al. 1994; Sonenshine 1991a; Wikel 1999). Some tick saliva components, such as apyrase or prostaglandins in high concentrations, inhibit platelet aggregation, thus blood clotting (Bowman et al. 1996; Ribeiro 1987). Other molecules have anti-inflammatory or immunosuppressive properties (reviewed in ref. Wikel 1999). By countering the host's innate and adaptive immune responses (Wikel 1996), the tick can feed for a longer duration (Barriga 1999). But the tick-host interaction is dynamic and is likely to be more complex than simply a contest between inflammatory factors of the host and anti-inflammatory factors of the tick. Evidence of this complexity was our finding in the hard tick A. americanum of the notably proinflammatory MIF, the first cytokine to be identified in an arthropod.

The tick MIF was discovered in a cDNA library produced from midgut tissues of partially fed female A. americanum ticks. A tick rather than a rabbit source for the cDNA clone was confirmed by identifying the gene's exons and introns in the genome and by using a tick MIF-specific anti-peptide antiserum to detect expression of the protein in tick tissues. Further evidence of the protein's tick origin was the finding that the deduced amino acid sequence was more similar to MIF proteins found in the parasitic nematodes than to mammalian MIF proteins.

Expression of the protein was documented in the salivary gland tissue as well as in midgut tissues of feeding and unfed A. americanum, but we did not detect its presence in hemolymph. Thus, the role of tick MIF in facilitating feeding remains to be determined. It is possible that tick MIF has a different function in A. americanum, and perhaps other arthropods, than in mammals. The mammalian MIF is known as a regulator of innate and acquired immunity, and has various roles from inducing inflammation in response to bacteria and viruses to activating macrophages and T-cells to release insulin from the pancreas (3ucala 2000). Here, the association with parasitism suggests a role in tick-host interaction. Proteins with MIF activity have been identified in parasitic helminths B. malayi, B. pahangi, T. spiralis, and Trichuris muris (Pastrana et al. 1998; Pennock et al. 1998). But the closest sequence in the C. elegans genome is only distantly related to MIF and this protein may not even have dopachrome tautomerase without immunomodulatory activity (Pennock et al. 1998). There was no evidence of an MIF homologue in the D. melanogaster genome. Tick MIF had dopachrome tautomerase activity; however, this activity was 10-fold less than that of human MIF. The importance of this enzymatic activity is unknown. All MIFs appear to have enzymatic activity in a catalytic site that is similar to that of the bacterial dopachrome tautomerase; however, MIFs have not been shown to interact with any of the substrates identified for these isomerases in biochemical studies (Swope et al. 1999).

The first invertebrate MIF homologue was identified in the filarial worm, Brugia malayi (Pastrana et al. 1998). Brugian MIF, like tick MIF, inhibited random macrophage migration to the same extent as did human MIF and was expressed in somatic tissues. The B. malayi MIF gene had two exons and a single 604 bp intron. The A. americanum MIF gene had three exons and two introns. The ORF for tick MIF was 348 nucleotides while B. malayi, human and mouse MIFs have ORFs of 345 nucleotides. The first exons of both A. americanum and B. malayi were lengths of 108 bp while first exons of human and mouse MIF genes were 107 bp in length. The human MIF gene had introns of 188 and 94 bp and the mouse gene had introns of 200 and 142 bp. These introns were considerably shorter than the introns of 647 and 1382 bp of A. americanum.

If tick MIF has a role in facilitating tick feeding, what could that role be MIF is a pro-inflammatory cytokine, principally by countering the immunosuppressive effects of glucocorticoids (Bucala 1996; Calandra et al. 1995). Thus, one possible role of tick MIF is to increase inflammation at the feeding site, although this would confound the suggestion that the principal role of salivary proteins is to reduce inflammation (Ribeiro 1987). An increase in blood flow that accompanies inflammation could benefit the tick, especially if other aspects of inflammation, such as pain, were inhibited by other tick products, such as an anaphylatoxin inactivator, as proposed by Ribeiro (Ribeiro 1987). Secretion of prostaglandins by ticks is associated with increased blood flow into the feeding lesion (Dickinson et al. 1979; Kemp et al. 1983; Madden et al. 1996), so the action of another tick product to increase inflammation is not inconceivable.

Another possible function of a tick MIF is to inhibit the migration of potentially dangerous macrophages toward the tick's mouthparts as it feeds or within the tick's midgut after feeding. Little is known about the physiology, biochemistry and molecular biology underlying the midgut epithelium. Little if any lumenal digestion occurs, and the lumen is at neutral pH (Smit et al. 1977). Most digestion occurs intracellularly, probably in lysosomes. Functional mammalian phagocytes, consumed by the tick with the blood meal, could be disruptive within its midgut. Sonenshine, writing about mammalian MIF, pointed out this cytokine could help contain macrophages to tick lesion site (Sonenshine 1991b). This could also occur with a tick MIF within the midgut.

As shown here, the tick MIF was not discernibly cross-reactive with antibodies to mouse or human MIF. Given the retained function of antibodies taken up in to midgut of A. americanum ticks (Jasinskas et al. 2000), an anti-MIF vaccine could be efficacious even against a molecule whose action is restricted to the midgut.

### EXAMPLE 2

DNA procedures. Female A. americanum ticks were fed for three days on rabbits, and mRNA from the ticks' midguts was used to produce a cDNA library in bacteriophage lambda and the inserts were subcloned into plasmid vectors as described (Jaworski et al. 1995). The laboratory-bred and -reared ticks were obtained from the Department of Entomology, Oklahoma State University. Inserts of these and other clones were sequenced completely in both directions by the fluorescent dideoxy termination method on an Applied Biosystems 377 automated sequencer (PE Biosystems). All sequences were initially analyzed through a BLASTX search of the Genbank database. Sequence alignments and bootstrapped neighbor-joining trees were produced with ClustalW (Higgins & Sharp 1988). For recombinant protein expression, the open reading frame of the cDNA was amplified with the forward primer 5′GCAATTCCATATGCCAACCCTTACAATTAACACG 3′ (SEQ ID NO:5), which contained a recognition site for NdeI and the first 24 nt of the tick MIF ORF (underlined) and the reverse primer 5′AAGCTTAGCCAGCAAAAGTTTTTCCGTTG 3′ (SEQ ID NO:6), which contained a recognition site for HindIII, a stop codon, and 22 nt of the tick MIF gene (underlined). The product was cloned first into the TA cloning vector (InVitrogen) and then into the expression vector pET23b (Novagen) and E. coli BL21 (DE3) cells. To identify and clone the gene from the genome, total DNA was isolated from A. americanum eggs that were pulverized over liquid nitrogen. The resultant powder was incubated in 0.5% SDS-100 mM Tris, pH 8.0-0.1 M EDTA with 20 μg/ml RNAase at 37° C. for 1 h and then in the same buffer with Proteinase K at 100 μg/ml at 50° C. for 3 h. The suspension was extracted with phenol and chloroform before ethanol precipitation. The above primers were used to amplify the tick MIF gene from the genome. Flanking sequences in the genome were obtained using the method of Devon et al. (Devon et al. 1995). Briefly, vectorette primers were prepared by duplexing the top strand (5′-GAATCGTAACCGTTCGTACGAGAATTCGTACGAGAATCGCTGTCCTCTCCA ACGAGCCAAGA-3′) (SEQ ID NO:7) and the bottom strand (5′-AGCTTCTTGGCTCGTTTTTTTTTGCAAAAA-3′) (SEQ ID NO:8) by mixing the oligonucleotides at 1.5×10⁻⁵ M each at 90° C. in 10 mM Tris, pH 7.5, 5 mM MgCl 2 and cooling at room temperature (RT). Total A. americanum DNA was digested with HindIII and ligated to 15-fold molar excess of vectorette primers for 5 hours (RT). PCR conditions were as follows: denaturation, 95° C., 30 s; annealing, for 1 min at 71° C. initially, decreasing by 2° C. to 55° C. per cycle; extension 72° C. for 2 min; then 95° C. for 30 s, annealing 55° C., extension at 720 C for 4 min, 30 cycles. In the primary reaction 1 ml of ligation product was amplified in 50 ml reaction using 40 pM MIF cDNA primer (for 5′-MIFgene flanking region: 5′-GTTCGCAGTAGTCTTCAGGAAGTC-3′ (SEQ ID NO:9) for 3′-MIF geneflanking region: 5′-CCAGCAAGTGATGTTGGCTAC-3′) (SEQ ID NO:10) and 40 pM vectorette primer (5′-CGAATCGTAACCGTTCGTACCAGAA-3′) (SEQ ID NO:11). Secondary PCR was performed using 1 ml of primary PCR product and 40 pM of each: internal MIF cDNA primers (for 5′-MIF gene flanking region: 5′-ATCTTGCTTGCGGGGATG-3′ (SEQ ID NO:12), for 3′MIF gene flanking region: 5′-CCAGCAAGTGATGTTGGCTAC-3′) (SEQ ID NO:13) and internal vectorette primer (5′-TCGTACGAGAATCGCTGTCCTCTC-3′) (SEQ ID NO:14).

Purification of MIF and protein procedures. Recombinant protein purification was carried out using a modification of the method of Bernhagen et al. for purifying MIF (Bernhagen et al. 1994). In brief, E. coli cells were grown in Luria-Bertani broth to an OD 600 of 0.6, and then protein synthesis was induced with isopropyl 1-thio-b-D-galactopyranoside for 2 h at 37° C. (Studier & Moffat 1986). Harvested cells were frozen, and the thawed pellets were sonicated. The lysate was filtered through a 0.22 mm membrane filter and then passed over a HiTrap Q anion-exchange column (Pharmacia) equilibrated with 50 mM Tris, pH 8.0. Recombinant protein was eluted with a continuous NaCl gradient from 0 to 500 mM on a BioRad low-pressure chromatography system. Fractions were examined with 10-20% Tris-tricine SDS-polyacrylamide gels (BioRad). Fractions with suspected MIF were pooled and further purified using a C18 reverse-phase liquid chromatography column (Vydec) on a LKB-Pharmacia high-pressure system, for which solvent A was 0.08% trifluoroacetic acid in H2O and solvent B was 0.08% trifluoroacetic acid in acetonitrile. Concentrated fractions were then diluted in 8 M urea-20 mM sodium phosphate, pH 7.2-5 mM dithiothreitol and then dialyzed against first 20 mM sodium phosphate, pH 7.2-5 mM DTT and then against 20 mM sodium phosphate buffer alone. The renatured tick MIF was filter-sterilized and stored at 4° C. until use. Protein concentrations were determined using a Bradford protein assay (Pierce). Edman degradation and automated cycle sequencing on a Hewlett Packard 1003 sequencer determined the N-terminal sequence of the purified protein. Mass spectroscopy of eluted peaks was done on the MALDI-TOF (Matrix Assisted Laser Desorption—Time of Flight) Voyager DE PRO (Perseptive Biosystems) using cinnapenic acid as a matrix. Mouse and human recombinant MIFs were prepared as described by Bernhagen et al. (1994). Tick peptide was synthesized by N-(9-flourenyl)-methoxycarbonyl chemistry using a Waters continuous flow semi-automatic instrument and purified by reverse-phase high performance liquid chromatography (Waters RPLC). The peptide was conjugated to keyhole limpet hemocyanin (KLH) using a Pierce conjugation kit.

Antisera. Adult female New Zealand White rabbits were immunized subcutaneously with 100 μg of a whole cell lysate of E. coli expressing recombinant protein, a synthetic tick peptide conjugated to keyhole limpet hemocyanin (KLH), or with KLH alone; the first doses were in complete Freund's adjuvant and two booster immunizations at 2-week intervals were in incomplete Freund's adjuvant. The rabbits were bled two weeks after the last immunization. The production of anti-mouse MIF polyclonal rabbit antiserum was described (Calandra et al. 1995).

Western blot analysis. Antigen preparations were prepared from ticks by dissecting female A. americanum salivary glands and midguts and then sonicating them on ice as described (Jaworski et al. 1990). As a control, Drosophila melanogaster were sonicated in the same way. Hemolymph was obtained as described (Jasinskas et al. 2000). SDS-polyacrylamide gel electrophoresis was performed with 10-20% Tris-tricine gradient gels on supernatants of the sonicates after centrifugation for 10 min at 12,000×g at room temperature. Gels were either stained with Coomassie blue or electrophoretically transferred to polyvinylidene difluoride (BioRad) or nitrocellulose membranes (Millipore). Blocking and incubation of the blots was carried out in 20 mM Tris, pH 7.4 with 150 mM NaCl, 5% nonfat milk, 0.2% Tween-20 and 3% horse serum. The antisera were diluted 1:500 in blocking buffer, and the binding of antibodies was detected with horseradish peroxidase-conjugated donkey anti-rabbit (Pierce) and EC achemiluminescent detection reagent (Amersham Pharmacia Biotech) and Kodak XR film.

Sandwich enzyme-linked immunosorbent assay. ELISA was performed by coating 96 well plates with monoclonal antibody to human MIF (Bacher et al. 1996). Briefly, samples were analyzed by MIF ELISA employing an anti-MIF capture mAb (XIV.14.3), a polyclonal rabbit anti-MIF detector antibody, and recombinant MIF as the standard. Purified recombinant human or tick MIF was added at an initial concentration of 1 mg/ml and serially diluted across the plate. Polyclonal rabbit antisera to mouse MIF or tick MIF were added each at a 1:250 final concentration. Rabbit antibody binding was detected with alkaline phosphatase-conjugated goat anti-rabbit immunoglobulin antiserum and p-nitrophenyl phosphate reactions were read at 405 nm.

In vitro functional assays. Isolation of human peripheral blood monocytes and performance of the assay was essentially as described (Pastrana et al. 1998). In brief, recombinant MIFs were treated with polymyxin B sepharose beads (BioRad) for 2 h at 4° C. to neutralize bacterial endotoxin. The Limulus amebocyte test (BioWhitaker) was used to confirm the absence of contaminating bacterial endotoxin in our samples. Human monocyte Chemotactic Protein 1 (MCP-1) was used as a stimulus of macrophage migration. Assays were performed in Geys medium (Gibco-BRL) with 0.2% low endotoxin BSA (Miles Laboratories) and 100 mM HEPES and in quadruplicate in 5 micron, 96-well micro-chemotaxis plates (Neuroprobe). The bottom portion of the plates was loaded with MCP-1 in the buffer or with buffer alone. Recombinant proteins and macrophages were placed in the top section, and the plates were incubated for 3 h at 37° C. Cells that migrated to the bottom wells were fixed and counted under 40× magnification by light microscopy. Results were compared by two-tailed Student's t-test. A semi-quantitative assay for dopachrome tautomerase activity was performed as previously described (Rosengren et al. 1997).

### REFERENCES

Bacher, M., Metz, C. N., Calandra, T., Mayer, K., Chesney, J., Lohoff, M., Gemsa, D., Donnelly, T. & Bucala, R. 1996 An essential regulatory role for macrophage migration inhibitory factor in T-cell activation. PNAS 93, 7849-7854.

Barriga, O. O. 1999 Evidence and mechanisms of immunosuppression in tick infestations. Genet Anal 15, 139-142.

Ben-Bassat, A., Bauer, K., Chang, S. Y., Myambo, K., Boosman, A. & Chang, S. 1987.

Processing of the initiation methionine from proteins: properties of the Escherichia coli methionine aminopeptidase and its gene structure. J Bacteriol 169, 751-757.

Bernhagen, J., Mitchell, R. A., Calandra, T., Voelter, W., Cerami, A. & Bucala, R. 1994 Purification, bioactivity, and secondary structure analysis of mouse and human macrophage migration inhibitory factor (MIF). Biochem 33, 14144-14155.

Bowman, A. S., Dillwith, J. W. & Sauer, J. R. 1996 Tick salivary prostaglandins: presence, origin and significance. Parasitol Today 12, 388-396.

Bucala, R. 2000 A most interesting factor. Nature 408, 146-147.

Bucala, R. 1996 MIF rediscovered: cytokine, pituitary hormone, and glucocorticoid-induced regulator of the immune response. FASEB J 10, 1607-1613.

Calandra, T., Bernhagen, J., Metz, C. N., Spiegel, L. A., Bacher, M., Donnelly, T., Cerami, A. & Bucala, R. 1995 MIF as a glucocorticoid-induced modulator of cytokine production. Nature 377, 68-71.

de Silva, A. M. & Fikrig, E. 1995 Growth and migration of Borrelia burgdorferi in Ixodes ticks during feeding. Am J of Trop Med Hyg 53, 397-404.

Devon, R. S., Porteous, D. J. & Brookes, A. J. 1995 Splinkerettes-improved vectorettes for greater efficiency in PCR walking. Nucleic Acids Research 23, 1644-1645.

Dickinson, R. G., O'Hagen, J. E., Schotz, M., Binnington, K. C. & Hegarty, M. P. 1979

Studies on the significance of smooth muscle contracting substances of the cattle tick, Boophilus microplus. J Aust Entomol Soc 18, 199-210.

Higgins, D. G. & Sharp, P. M. 1988 CLUSTAL: A Package for Performing Multiple Sequence Alignment on a Microcomputer. Gene 73, 237-234.

Jasinskas, A., Jaworski, D. C. & Barbour, A. G. 2000 Amblyomma americanum: specific uptake of immunoglobulins into tick hemolymph during feeding. Exp. Parasitol. 96, 213-221.

Jaworski, D. C., Muller, M. T., Simmen, F. A. & Needham, G. R. 1990 Amblyomma americanum: Identification of tick salivary gland antigens from unfed and early feeding females with comparisons to Ixodes dammini and Dermacentor variabilis. Exp Parasitol 70, 217-226.

Jaworski, D. C., Simmen, F. A., Lamoreaux, W., Coons, L. B., Muller, M. T. & Needham, G. R. 1995 A secreted calreticulin protein in ixodid tick saliva. J Insect Physiol 41.

Kemp, D. H., Hales, J. R., Schleger, A. V. & Fawcett, A. A. 1983 Comparison of cutaneous hyperemia in cattle elicited by larvae of Boophilus microplus and by prostaglandins and other mediators. Experentia 39, 725-727.

Madden, R. D., Sauer, J. R., Dillwirth, J. W. & Bowman, A. S. 1996 Dietary modification of host blood lipids affect reproduction in the lone star tick, Amblyomma americanum. J Parasitol 82, 203-209.

Nuttall, P. A. 1998 Displaced tick-parasite interactions at the host interface. Parasitology 116, S65-S72.

Pastrana, D. V., Raghavan, N., Fitzgerald, P., Eisinger, S. W., Metz, C., Bucala, R., Schleimer, R. P., Bickel, C. & Scott, A. L. 1998 Filarial nematode parasites secrete a homologue of the human cytokine macrophage migration inhibitory factor. Infect & Immun 66, 5955-5963.

Pennock, J. L., Behnke, J. M., Bickle, Q. D., Devaney, E., Grencis, R. K., Isaac, R. E., Jo, M. E., Zhang, Y. & Meyer, D. J. 1998 Rapid purification and characterization of L-dopachrome-methyl ester tautomerase (macrophage-migration-inhibitory factor) Trichinella spiralis, Trichuris muris and Brugia pahangi. Biochem J 335, 495-498.

Ribeiro, J. M. C. 1987 Role of saliva in blood-feeding by arthropods. Ann Rev Entomol 32,463-478.

Rosengren, E., Aman, P., Thelin, S., Hansson, C., Ahlfors, S., Bjork, P., Jacobsson, L. & Rorsman, H. 1997 The macrophage migration inhibitory factor MIF is phenylpyruvate tautomerase. FEBS Letters 417, 85-88.

Sauer, J. R., McSwain, J. L. & Essenberg, R. C. 1994 Cell membrane receptors and regulation of cell function in ticks and blood-sucking insects. Intl J Parasit 24, 33-52.

Smit, J. D. G., Grandjean, O., Guggenheim, R. & Winterhalter, K. H. 1977 Hemoglobin crystals in the midgut of Omithodoros moubata Murray. Nature 266, 536-538.

Sonenshine, D. 1991a The Biology of Ticks, Volume 1.

Sonenshine, D. 1991b The Biology of Ticks, Volume 2.

Studier, F. W. & Moffat, B. A. 1986 Use of bacteriophage T7 RNA polymerase to direct selective high-level expression of cloned genes. J Mol Biol 189, 113-130.

Swope, M., Sun, H. W., Blake, P. R. & Lolis, E. 1998 Direct link between cytokine activity and a catalytic site for macrophage migration inhibitory factor. EMBO J 17, 3534-3541.

Swope, M., and Lolis, E. 1999 Macrophage migration inhibitory factor: cytokine, hormone, or enzyme Rev. Physiol. Biochem. Pharmacol. 139, 1-32.

Trager, W. 1939 Acquired immunity to ticks. J. Parasit 25, 57-81.

Walker, D. H., Barbour, A. G., Oliver, J. H., Lane, R. S., Dumler, J. S., Dennis, D. T., Persing, D. H., Azad, A. F. & McSweegan, E. 1996 Emerging bacterial zoonotic and vector-borne diseases. Ecological and epidemiological factors. Jama 275, 463-9.

Wikel, S. 1999 Tick modulation of host immunity: an important factor in pathogen transmission. Intl J Parasitol 29, 851-859.

Wikel, S. K. 1996 Host immunity to ticks. Ann Rev Entomol 41, 1-22.

Wikel, S. K. & Bergman, D. 1997 Tick-Host Immunology: Significant advances and challenging opportunities. Parasitol Today 13, 383-389.

TABLE 1
Recombinant tick MIF and human MIF in a
random macrophage migration assay

Mean no. migrating

| | | | | |
|---|---|---|---|---|
| Sample¹ | ng/ml | cells | (95% CI)² | p³ |
| Control | — | 19.0 | (14.6-23.5) | — |
| Tick MIF | 10 | 14.0 | (10.5-17.5) | 0.15 |
| Human MIF | 10 | 13.7 | ( 9.8-17.7) | 0.11 |
| Tick MIF | 100 | 10.0 | ( 8.6-11.5) | 0.003 |
| Human MIF | 100 | 10.9 | ( 9.5-12.3) | 0.003 |
| MCP-1⁴ | 20 | 39.4 | (23.8-55.1) | 0.03 |

¹Performed in quadruplicate.
²Mean number of cells per 40x field with 95% confidence intervals (CI).
³Two-tailed student's t-test.
⁴Monocyte Chemotactic Protein 1.

Although the invention has been described with reference to the above examples, it will be understood that modifications and variations are encompassed within the spirit and scope of the invention. Accordingly, the invention is limited only by the following claims.

1. A substantially pure polypeptide characterized as having an amino acid sequence comprising amino acid residues CLSPKENKKHSAVLFEHIEKTL (SEQ ID NO:3) and a molecular weight of about 12 kD.

2. The polypeptide of claim 1, wherein the polypeptide has an amino acid sequence as set forth in SEQ ID NO:2.

3. A substantially pure polypeptide having an amino acid sequence that is about 60% homologous to a polypeptide of claim 2.

4. A substantially pure polypeptide having an amino acid sequence that is about 70% homologous to a polypeptide of claim 2.

5. A substantially pure polypeptide having an amino acid sequence that is about 80% homologous to a polypeptide of claim 2.

6. A substantially pure polypeptide having an amino acid sequence that is about 90% homologous to a polypeptide of claim 2.

7. A substantially pure polypeptide comprising the contiguous amino acid sequence CLSPKENKKHSAVLFEHIEKTL (SEQ ID NO:3).

8. An isolated polynucleotide encoding a polypeptide as in claims 1 or 7.

9. An isolated peptide having the sequence CLSPKENKKHSAVLFEHIEKTL (SEQ ID NO:3).

10. An isolated polynucleotide encoding a peptide having an amino acid sequence CLSPKENKKHSAVLFEHIEKTL (SEQ ID NO:3).

11. An isolated polynucleotide selected from the group consisting of:
(a) a polynucleotide encoding a polypeptide having an amino acid sequence as set forth in SEQ ID NO:2;
(b) a polynucleotide of (a), wherein T can be U;
(c) a polynucleotide complementary to (a) or (b);
(d) a polynucleotide having a nucleotide sequence as set forth in SEQ ID NO:1; and
(e) degenerate variants of (a), (b), (c) or (d).

12. An isolated polynucleotide having at least 15 continuous base pairs that hybridizes to a polynucleotide encoding a polypeptide as set forth in SEQ ID NO:2 selected from the group consisting of:
(a) a polynucleotide encoding a polypeptide having an amino acid sequence as set forth in SEQ ID NO:2;
(b) a polynucleotide of (a), wherein T can be U;
(c) a polynucleotide complementary to (a) or (b);
(d) a polynucleotide having a nucleotide sequence as set forth in SEQ ID NO:1 or SEQ ID NO:3; and
(e) degenerate variants of (a), (b), (c) or (d).

13. An isolated polynucleotide at least 15 bases in length which hybridizes under moderately to highly stringent conditions to DNA encoding a polypeptide as set forth in SEQ ID NO:2.

14. An antibody that binds to a polypeptide of any of claims 1 or 9 or binds to immunoreactive fragments thereof.

15. The antibody of claim 14, wherein the antibody is polyclonal.

16. The antibody of claim 14, wherein the antibody is monoclonal.

17. An expression vector comprising a polynucleotide of claim 1.

18. The expression vector of claim 17, wherein the vector is virus-derived.

19. The expression vector of claim 17, wherein the vector is plasmid-derived.

20. A host cell comprising a vector of claim 17.

21. A method of producing tick MIF polypeptide comprising:
(a) expressing a polynucleotide encoding the polypeptide of claim 1 in a host cell; and
(b) recovering the MIF polypeptide.

22. An isolated polynucleotide sequence according to claim 8, wherein said polynucleotide sequence encodes a polypeptide that produces an immune response against tick infestation in a host when said polynucleotide is administered to and expressed in said host.

23. A host cell according to claim 20, wherein said cell is a bacterial, yeast, mammalian or insect cell.

24. A method of inducing an immune response to a tick polypeptide in a subject comprising administering to the subject a pharmaceutical composition containing an immunogenically effective amount of isolated MIF protein characterized as having an amino acid sequence comprising amino acid residues CLSPKENKKHSAVLFEHIEKTL (SEQ ID NO:3) and a molecular weight of about 12 kD.

25. The method of claim 24, wherein said tick carries a pathogen selected from the group consisting of Borrelia sp., Theileria sp., Ehrlichia sp., Babesia sp., Rickettsia sp. and tick-borne encephalitis virus.

26. The method of claim 24, wherein said protein is in a pharmaceutically acceptable carrier.

27. The method of claim 25, wherein said pharmaceutically acceptable carrier contains an adjuvant.

28. The method of claim 24, wherein the subject is a mammal.

29. The method of claim 28, wherein the mammal is a human.

30. The method of claim 24, wherein the subject is a bovine, porcine, ovine, avian, feline, canine, equine, murine, cervine, caprine, lupine, or leporidine species.

31. A pharmaceutical composition useful for inducing an immune response to a tick in an animal comprising an immunogenically effective amount of an isolated MIF protein characterized as having an amino acid sequence comprising amino acid residues CLSPKENKKHSAVLFEHIEKTL (SEQ ID NO:3) and a molecular weight of about 12 kD, in a pharmaceutically acceptable carrier.

32. The pharmaceutical composition of claim 31, wherein the pharmaceutically acceptable carrier contains an adjuvant.

33. A method of inducing an immune response to a tick polypeptide in a subject comprising administering to the subject a pharmaceutical composition containing an immunogenically effective amount of isolated MIF antibody that binds to a protein characterized as having an amino acid sequence comprising amino acid residues CLSPKENKKHSAVLFEHIEKTL (SEQ ID NO:3) and a molecular weight of about 12 kD.

34. A kit useful for the detection of tick MIF polynucleotide, the kit comprising a carrier means with at least two containers, wherein the first container contains a nucleic acid which encodes the amino acid sequence of SEQ ID NO:2 or a nucleic acid probe at least 15 bases in length that hybridizes with a nucleic acid sequence that encodes SEQ ID NO:2 or SEQ ID NO:3, and wherein a second container contains a label for detection of nucleic acid for identification of the presence of tick MIF polynucleotide.

35. The kit of claim 34, wherein the label is selected from the group consisting of a radioisotope, a bioluminescent compound, a chemiluminescent compound, a fluorescent compound, a metal chelate, or an enzyme.

36. A method for detecting antibody to tick MIF polypeptide in a sample comprising contacting the sample with tick MIF polypeptide, or fragments thereof, under conditions which allow the antibody to bind to tick MIF polypeptide and detecting the binding of the antibody to the tick MIF polypeptide, or fragments thereof.

37. The method of claim 36, wherein the tick MIF polypeptide is detectably labeled.

38. A kit useful for the detection of tick MIF polypeptide, the kit comprising carrier means containing one or more containers comprising a first container containing a tick MIF binding reagent.

39. The kit of claim 38, wherein the reagent is an antibody.

40. The kit of claim 38, wherein the antibody is monoclonal.

41. A kit useful for the detection of antibody to tick MIF polypeptide, the kit comprising carrier means containing one or more containers comprising a first container containing tick MIF polypeptide.

42. A method for identifying a compound which binds to a polypeptide of claim 1 comprising:
(a) incubating components comprising the compound and the polypeptide under conditions sufficient to allow the components to interact; and
(b) measuring the binding of the compound to the polypeptide.

43. The method of claim 42, wherein the compound is a peptide.

44. The method of claim 42, wherein the compound is a peptidomimetic.

45. The method of claim 42, wherein the compound is an antibody.

46. A method for accelerating wound healing in a subject in need of such treatment comprising contacting the site of the wound with a therapeutically effective amount of a composition containing a polypeptide of claim 1.

47. A method for treating a tumor or a cell proliferative disease in a subject in need of such treatment comprising contacting the site of the tumor or contacting the subject with a therapeutically effective amount of a composition containing a polypeptide of claim 1.
